SEQUENCE LISTING (69 sequences)

SEQ ID NO: 1 - protein, 367 aa, Rhizopus oryzae
  PROPEP 1..98
  PEPTIDE 99..367
MVPVSGKSGSSTTAVSASDNSALPPLISSRCAPPSNKGSKSDLQAEPYYMQKNTEWYESHGGNLTSIGKRDDNLVGGMTLDLPSDAPPISLSGSTNSASDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIFTVGGPRVGNPTFAYYVESTGIPFQRTVHKRDIVPHVPPQSFGFLHPGVESWIKSGTSNVQICTSEIETKDCSNSIVPFTSLLDHLSYFDINEGSCL

SEQ ID NO: 2 - protein, 269 aa, Rhizopus oryzae
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIFTVGGPRVGNPTFAYYVESTGIPFQRTVHKRDIVPHVPPQSFGFLHPGVESWIKSGTSNVQICTSEIETKDCSNSIVPFTSLLDHLSYFDINEGSCL

SEQ ID NO: 3 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIFTVGGPRVGNPTFAYYVCSTGIPFQRTVHKRDIVPHLPPQSFGFLHPGVESWIKSGTSNVQICTSCIETKDCSNSIVPFTSLLDHLSYFGINEGSCL

SEQ ID NO: 4 - protein, 269 aa, synthetic construct
SDGGKVAEATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIFTVGGPRVGNPTFAYYVCSTGIPFQRTVHKRDIVPHLPPQSFGFLHPGVESWIKSGTSNVQICTSCIETKDCSNSIVPFTSLLDHLSYFGINEGSCL

SEQ ID NO: 5 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLNSYEQVVNDYFPVIQEQLTANPTYKVIVTGHSLGGAQALLAGMDLYQREPRLSPKNLSIFTVGGPRVGNPTFAYYVCSTGIPFQRTVHKRDIVPHLPPQSFGFLHPGVESWIKSGTSNVQICTSCIETKDCSNSIVPFTSLLDHLSYFGINEGSCL

SEQ ID NO: 6 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTAYPTYKVIVTGHSLGGAQALLAGMDLYQREPRLSPKNLSIFTVGGPRVGNPTFAYYVCSTGIPFQRTVHKRDIVPHLPPQSFGFLHPGVESWIKSGTSNVQICTSCIETKDCSNSIVPFTSLLDHLSYFGINEGSCL

SEQ ID NO: 7 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPDYKVIVTGHSLGGAQALLAGMDLYQREPRLSPKNLSIFTVGGPRVGNPTFAYYVCSTGIPFQRTVHKRDIVPHLPPQSFGFLHPGVESWIKSGTSNVQICTSCIETKDCSNSIVPFTSLLDHLSYFGINEGSCL

SEQ ID NO: 8 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIYTVGGPRVGNPTFAYYVCSTGIPFQRTVHKRDIVPHLPPQSFGFLHPGVESWIKSGTSNVQICTSCIETKDCSNSIVPFTSLLDHLSYFGINEGSCL

SEQ ID NO: 9 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIFTVGGPRVGNPTFANYVCSTGIPFQRTVHKRDIVPHLPPQSFGFLHPGVESWIKSGTSNVQICTSCIETKDCSNSIVPFTSLLDHLSYFGINEGSCL

SEQ ID NO: 10 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIFTVGGPRVGNPTFAYYVCSTKIPFQRTVHKRDIVPHLPPQSFGFLHPGVESWIKSGTSNVQICTSCIETKDCSNSIVPFTSLLDHLSYFGINEGSCL

SEQ ID NO: 11 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIFTVGGPRVGNPTFAYYVCSTGIPFHRTVHKRDIVPHLPPQSFGFLHPGVESWIKSGTSNVQICTSCIETKDCSNSIVPFTSLLDHLSYFGINEGSCL

SEQ ID NO: 12 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIFTVGGPRVGNPTFAYYVCSTGIPFYRTVHKRDIVPHLPPQSFGFLHPGVESWIKSGTSNVQICTSCIETKDCSNSIVPFTSLLDHLSYFGINEGSCL

SEQ ID NO: 13 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIFTVGGPRVGNPTFAYYVCSTGIPFQRTVHKRDIVPHLPPQSFGFLHPGVESWIKSDTSNVQICTSCIETKDCSNSIVPFTSLLDHLSYFGINEGSCL

SEQ ID NO: 14 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIFTVGGPRVGNPTFAYYVCSTGIPFQRTVHKRDIVPHLPPQSFGFLHPGVESWIKSGTSNVQICTSCIETKDCSNSIVPFTSLLDHLSYFGINEGLCL

SEQ ID NO: 15 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIYTVGGPRVGNPTFAYYVCSTGIPFHRTVHKRDIVPHLPPQSFGFLHPGVESWIKSGTSNVQICTSCIETKDCSNSIVPFTSLLDHLSYFGINEGLCL

SEQ ID NO: 16 - protein, 269 aa, synthetic construct
SDGGKVRAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIYTVGGPRVGNPTFAYYVCSTGIPFHRTVHKRDIVPHLPPQSFGFLHPGVESWIKSGTSNVQICTSCIETKDCSNSIVPFTSLLDHLSYFGINEGLCL

SEQ ID NO: 17 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFYSSYEQVVNDYFPVIQEQLTANPTYKVIVTGHSLGGAQALLAGMDLYQREPRLSPKNLSIYTVGGPRVGNPTFAYYVCSTGIPFHRTVHKRDIVPHLPPQSFGFLHPGVESWIKSGTSNVQICTSCIETKDCSNSIVPFTSLLDHLSYFGINEGLCL

SEQ ID NO: 18 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAFALLAGMDLYQREPRLSPKNLSIYTVGGPRVGNPTFAYYVCSTGIPFHRTVHKRDIVPHLPPQSFGFLHPGVESWIKSGTSNVQICTSCIETKDCSNSIVPFTSLLDHLSYFGINEGLCL

SEQ ID NO: 19 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAHALLAGMDLYQREPRLSPKNLSIYTVGGPRVGNPTFAYYVCSTGIPFHRTVHKRDIVPHLPPQSFGFLHPGVESWIKSGTSNVQICTSCIETKDCSNSIVPFTSLLDHLSYFGINEGLCL

SEQ ID NO: 20 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAAMDLYQREPRLSPKNLSIYTVGGPRVGNPTFAYYVCSTGIPFHRTVHKRDIVPHLPPQSFGFLHPGVESWIKSGTSNVQICTSCIETKDCSNSIVPFTSLLDHLSYFGINEGLCL

SEQ ID NO: 21 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLFIYTVGGPRVGNPTFAYYVCSTGIPFHRTVHKRDIVPHLPPQSFGFLHPGVESWIKSGTSNVQICTSCIETKDCSNSIVPFTSLLDHLSYFGINEGLCL

SEQ ID NO: 22 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIYTFGGPRVGNPTFAYYVCSTGIPFHRTVHKRDIVPHLPPQSFGFLHPGVESWIKSGTSNVQICTSCIETKDCSNSIVPFTSLLDHLSYFGINEGLCL

SEQ ID NO: 23 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIYTVGCPRVGNPTFAYYVCSTGIPFHRTVHKRDIVPHLPPQSFGFLHPGVESWIKSGTSNVQICTSCIETKDCSNSIVPFTSLLDHLSYFGINEGLCL

SEQ ID NO: 24 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIYTVGGPRVGNPTFAYYVCSTGIPFHRVVHKRDIVPHLPPQSFGFLHPGVESWIKSGTSNVQICTSCIETKDCSNSIVPFTSLLDHLSYFGINEGLCL

SEQ ID NO: 25 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIYTVGGPRVGNPTFAYYVCSTGIPFHRTVHKRDIVPHLPPQSFGYLHPGVESWIKSGTSNVQICTSCIETKDCSNSIVPFTSLLDHLSYFGINEGLCL

SEQ ID NO: 26 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIYTVGGPRVGNPTFAYYVCSTGIPFHRTVHKRDIVPHLPPQSFGFLHPGVEYWIKSGTSNVQICTSCIETKDCSNSIVPFTSLLDHLSYFGINEGLCL

SEQ ID NO: 27 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIYTVGGPRVGNPTFAYYVCSTGIPFHRTVHKRDIVPHLPPQSFGFLHPGVESWIKSGTSNVQICTSCIETKDCSNSIVPFTSLLDHLSYYGINEGLCL

SEQ ID NO: 28 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIYTFGCPRVGNPTFAYYVCSTGIPFHRTVHKRDIVPHLPPQSFGYLHPGVESWIKSGTSNVQICTSCIETKDCSNSIVPFTSLLDHLSYFGINEGLCL

SEQ ID NO: 29 - DNA, 31 bp, synthetic construct
  source 1..31: mol_type = other DNA
ctggtctcggatggtgcccgttagcggaaag

SEQ ID NO: 30 - DNA, 37 bp, synthetic construct
  source 1..37: mol_type = other DNA
ctggtctcgtgtaggttgggttagcagtcaactgctc

SEQ ID NO: 31 - DNA, 31 bp, synthetic construct; identical to SEQ ID NO: 29
  source 1..31: mol_type = other DNA

SEQ ID NO: 32 - DNA, 37 bp, synthetic construct
  source 1..37: mol_type = other DNA
ctggtctcgtgtaggttggataagcagtcaactgctc

SEQ ID NO: 33 - DNA, 31 bp, synthetic construct; identical to SEQ ID NO: 29
  source 1..31: mol_type = other DNA

SEQ ID NO: 34 - DNA, 37 bp, synthetic construct
  source 1..37: mol_type = other DNA
ctggtctcgtgtaatctgggttagcagtcaactgctc

SEQ ID NO: 35 - DNA, 31 bp, synthetic construct; identical to SEQ ID NO: 29
  source 1..31: mol_type = other DNA

SEQ ID NO: 36 - DNA, 37 bp, synthetic construct
  source 1..37: mol_type = other DNA
ctggtctcgtgtaatctggataagcagtcaactgctc

SEQ ID NO: 37 - DNA, 37 bp, synthetic construct
  source 1..37: mol_type = other DNA
ctggtctcgtacaaggttatcgttaccggtcactctc

SEQ ID NO: 38 - DNA, 33 bp, synthetic construct
  source 1..33: mol_type = other DNA
ctggtctcggaatgggataccagtggagcaaac

SEQ ID NO: 39 - DNA, 37 bp, synthetic construct; identical to SEQ ID NO: 37
  source 1..37: mol_type = other DNA

SEQ ID NO: 40 - DNA, 33 bp, synthetic construct; identical to SEQ ID NO: 38
  source 1..33: mol_type = other DNA

SEQ ID NO: 41 - DNA, 40 bp, synthetic construct
  source 1..40: mol_type = other DNA
ctggtctcgattccagagaaccgttcacaagagagacatc

SEQ ID NO: 42 - DNA, 36 bp, synthetic construct
  source 1..36: mol_type = other DNA
ctggtctcggctacaaacaggaaccctcgttaatac

SEQ ID NO: 43 - DNA, 40 bp, synthetic construct
  source 1..40: mol_type = other DNA
ctggtctcgattccatagaaccgttcacaagagagacatc

SEQ ID NO: 44 - DNA, 36 bp, synthetic construct; identical to SEQ ID NO: 42
  source 1..36: mol_type = other DNA

SEQ ID NO: 45 - DNA, 40 bp, synthetic construct
  source 1..40: mol_type = other DNA
ctggtctcgattctacagaaccgttcacaagagagacatc

SEQ ID NO: 46 - DNA, 36 bp, synthetic construct; identical to SEQ ID NO: 42
  source 1..36: mol_type = other DNA

SEQ ID NO: 47 - DNA, 40 bp, synthetic construct; identical to SEQ ID NO: 41
  source 1..40: mol_type = other DNA

SEQ ID NO: 48 - DNA, 45 bp, synthetic construct
  source 1..45: mol_type = other DNA
ctggtctcggctacaaacacaaaccctcgttaataccgaagtagg

SEQ ID NO: 49 - DNA, 40 bp, synthetic construct; identical to SEQ ID NO: 43
  source 1..40: mol_type = other DNA

SEQ ID NO: 50 - DNA, 45 bp, synthetic construct; identical to SEQ ID NO: 48
  source 1..45: mol_type = other DNA

SEQ ID NO: 51 - DNA, 40 bp, synthetic construct; identical to SEQ ID NO: 45
  source 1..40: mol_type = other DNA

SEQ ID NO: 52 - DNA, 45 bp, synthetic construct; identical to SEQ ID NO: 48
  source 1..45: mol_type = other DNA

SEQ ID NO: 53 - DNA, 35 bp, synthetic construct
  source 1..35: mol_type = other DNA
ctggtctcggatggttccagtttctggtaagtctg

SEQ ID NO: 54 - DNA, 33 bp, synthetic construct
  source 1..33: mol_type = other DNA
ctggtctcgttggaccaccaacggtgtagatgg

SEQ ID NO: 55 - DNA, 35 bp, synthetic construct; identical to SEQ ID NO: 53
  source 1..35: mol_type = other DNA

SEQ ID NO: 56 - DNA, 47 bp, synthetic construct
  source 1..47: mol_type = other DNA
ctggtctcgttggaccaccaaaggtgtagatggacaagttctttggg

SEQ ID NO: 57 - DNA, 35 bp, synthetic construct; identical to SEQ ID NO: 53
  source 1..35: mol_type = other DNA

SEQ ID NO: 58 - DNA, 47 bp, synthetic construct
  source 1..47: mol_type = other DNA
ctggtctcgttggacaaccaacggtgtagatggacaagttctttggg

SEQ ID NO: 59 - DNA, 35 bp, synthetic construct; identical to SEQ ID NO: 53
  source 1..35: mol_type = other DNA

SEQ ID NO: 60 - DNA, 47 bp, synthetic construct
  source 1..47: mol_type = other DNA
ctggtctcgttggacaaccaaaggtgtagatggacaagttctttggg

SEQ ID NO: 61 - DNA, 33 bp, synthetic construct
  source 1..33: mol_type = other DNA
ctggtctcgccaagagtcggtaacccaactttc

SEQ ID NO: 62 - DNA, 45 bp, synthetic construct; identical to SEQ ID NO: 48
  source 1..45: mol_type = other DNA

SEQ ID NO: 63 - DNA, 33 bp, synthetic construct; identical to SEQ ID NO: 61
  source 1..33: mol_type = other DNA

SEQ ID NO: 64 - DNA, 45 bp, synthetic construct; identical to SEQ ID NO: 48
  source 1..45: mol_type = other DNA

SEQ ID NO: 65 - protein, 269 aa, synthetic construct
SDGGKVAAATTAQIQEFTKYAGIAATAYCRSVVPGNKWDCVQCLKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIYTFGCPRVGNPTFAYYVCSTGIPFHRTVHKRDIVPHLPPQSFGYLHPGVESWIKSGTSNVQICTSCIETKDCSNSIVPFTSLLDHLSYFGINEGLCL

SEQ ID NO: 66 - protein, 269 aa, synthetic construct
SDGGKVAAATTAQIQEFTKYAGIAATAYCRSVVPGNKWDCVQCQKWVPDGKIITTFTSLLSDTNGYVLRSDGQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIYTFGCPRVGNPTFAYYVCSTGIPFHRTVHKRDIVPHLPPQSFGYLHPGVESWIKSGTSNVQICTSCIETKDCSNSIVPFTSLLDHLSYFGINEGLCL

SEQ ID NO: 67 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIYTFGCPRVGNPTFAYYVCSTGIPFHRTVHKRDIVPHLPPQSFGYLHPGVESWIVSGTSNVQICTSCIETKDCSNSIVPFTSLLDHLSYFGINEGLCL

SEQ ID NO: 68 - protein, 269 aa, synthetic construct
SDGGKVAAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIYTFGCPRVGNPTFAYYVCSTGIPFHRTVHKRDIVPHLPPQSFGYLHPGVESWIKSGTSNVQICTSCIETKYCSNSIVPFTSLLDHLSYFGINEGLCL

SEQ ID NO: 69 - protein, 269 aa, synthetic construct
SDGGKVAAATTAQIQEFTKYAGIAATAYCRSVVPGNKWDCVQCLKWVPDGKIITTFTSLLSDTNGYVLRSDGQKTIYLVFRGTNSFRSAITDIVFNFSDYKPVKGAKVHAGFLSSYEQVVNDYFPVIQEQLTANPTYKVIVTGHSLGGAQALLAGMDLYQREPRLSPKNLSIYTFGCPRVGNPTFAYYVCSTGIPFHRTVHKRDIVPHLPPQSFGYLHPGVESWIVSGTSNVQICTSCIETKYCSNSIVPFTSLLDHLSYFGINEGLCL

(19) United States
(12) Patent Application Publication
(10) Pub. No.: US 2025/0136958 A1
(43) Pub. Date: May 1, 2025

(54) ENGINEERING OF RHIZOPUS ORYZAE LIPASE TO INCREASE ITS THERMOSTABILITY FOR THE PRODUCTION OF STRUCTURED TRIACYLGLYCEROLS

(71) Applicant: WILMAR INTERNATIONAL LIMITED, Singapore (SG)

(72) Inventors: Jeng Yeong CHOW, Singapore (SG); Nguyen Kien Truc GIANG, Singapore (SG)

(21) Appl. No.: 18/834,807

(22) PCT Filed: Feb. 9, 2023
(86) PCT No.: PCT/SG2023/050069

§ 371 Date: Jul. 31, 2024

(30) Foreign Application Priority Data
Feb. 18, 2022 (SG) 10202201603Y

(51) Int. Cl. C12N 9/20; C12N 15/81; C12P 7/649

(52) U.S. Cl. CPC C12N 9/20; C12N 15/81; C12P 7/649; C12Y 301/01003

(57) ABSTRACT

Described herein are modified enzymes with increased thermostability. The modified enzymes of SEQ ID NO: 3 includes at least one amino acid substitution of SEQ ID NO: 3, the at least one amino acid substitution selected from the group consisting of A8E, S114N, N134Y, T136D, F173Y, Y187N, G193K, Q197H, Q197Y, G228D, S267L, A7R, L113Y, Q150F, Q150H, G155A, S171F, V175F, G177C, T199V, F216Y, S223Y, F261Y, and any combinations thereof. The modified enzymes may be used in methods of forming a first triglyceride by mixing a second triglyceride, a first fatty acid, and the modified enzyme to form the first triglyceride which is a mixture of fatty acids from the second triglyceride and the first fatty acid. Biodiesel may be produced by mixing a fatty acid or a triglyceride, a short-chained alcohol, and the modified enzyme to form a fatty acid ester of the short-chained alcohol.

The present application claims priority to Singapore patent application number 10202201603Y filed on 18 Feb. 2022 titled “Engineering of ROL lipase to increase its thermostability for the production of structured TAGs” which is incorporated by reference herein in its entirety. A sequence listing in xml format of the amino acid sequences and nucleotide sequences described herein is provided with the present application and is incorporated herein by reference.

### TECHNICAL FIELD

The present invention relates to modified enzymes, in particular modified lipases.

### BACKGROUND

1,3-regiospecific lipases are important enzymes that are heavily utilized in the food industries to produce structured triacylglycerols (TAGs) (may also be termed triglyceride). The Rhizopus oryzae lipase (ROL) (may also be known as Rhizopus arrhizus lipase) has recently gained traction to be used in various applications due to its high selectivity and catalytic efficiency. The wild type ROL has a peptide sequence as shown in SEQ ID NO: 1 with a propeptide portion (amino acid residues 1 to 98). The mature peptide of ROL starts from amino acid residue 99 of SEQ ID NO: 1 and is also shown in SEQ ID NO: 2. However, its low thermostability limits its use towards reactions that work at lower temperature (i.e. it cannot be used with reactions that require higher temperatures). For example, the enzyme cannot be used for the production of 1,3-dioleoyl-2-palmitoylglycerol (OPO) and 1,3-stearoyl-2-oleoyl-glycerol (SOS) due to the high melting points of these substrates. Despite various engineering efforts used to improve the thermostability of ROL, the enzyme is unable to function at temperatures above 60° C.

Rhizomucor miehei lipase (RML) and Rhizopus oryzae lipase (ROL) are 1,3-regioselective lipases that are widely used in the food industry to produce structured TAGs. These enzymes can target fatty acids on the sn-1 and sn-3 positions of a TAG molecule while leaving the fatty acid on the sn-2 position intact, allowing the production of TAGs with unique functions and nutritional values. An example of an important structured TAG is 1,3-dioleoyl-2-palmitoylglycerol (OPO), which is found in the human milk that can be easily absorbed by infants to provide them with the essential nutrients for the development of cognitive and visual functions. Production of OPO through the enzymatic interesterification (EIE) of plant oil has increased in recent years due to the increased demand of using OPO in infant milk formulations.¹ Another important use of 1,3-regioselective lipases is the production of 1,3-stearoyl-2-oleoyl-glycerol (SOS), which can be used as a cocoa butter equivalent (CBE). The chemical composition of CBE resembles the composition of fats found in cocoa butter and hence CBE is widely used as a chocolate fat mimetic.²

In recent years, ROL has emerged as an important lipase for industrial applications due to its higher specific activity and 1,3-regioselectivity, in comparison with RML.³ However, ROL has a lower thermostability and this limits the use of ROL to reactions that can occur at lower temperature. To increase the thermostability of ROL, Kohno et al used error-prone PCR and a high-throughput halo assay to identify a ROL E218V mutant with a 15° C. increase in thermostability.⁴ More recently, Zhao et al used a combination of multiple sequence alignment and computation tools to design a ROL variant with an additional disulfide bond at the E190 and E238 positions. This quadruple mutant (V209L/D262G/E190C/E238C) (SEQ ID NO: 3) can retain about 50% of its activity at 55° C. for 1 day.⁵ Although these engineering efforts were able to increase the thermostability of ROL, most of these ROL variants are still not suitable for EIE reactions that occur at even higher temperature. For example, production of OPO and SOS will require the reaction to reach 60° C. and 70° C., respectively, due to the high melting points of the substrates used in the reaction. Production of 1-oleoyl-2-palmitoyl-3-linoleoylglycerol (OPL) also require similar high temperatures.

### SUMMARY

Herein is described the engineering of ROL to further increase its thermostability so that the enzyme can be used in EIE reactions at temperature 60° C. and higher and the screening of ROL mutants to identify variants that can retain its activity at temperature 60° C. and higher. In particular, the ROL-10× mutant obtained after two rounds of mutagenesis and screening retains most of its activity at 70° C. and may be used in industrial EIE reactions to produce OPO, SOS, and OPL. The thermostability of a developed mutant ROL-10× was further improved by B-factor site-saturation mutagenesis.

In a first aspect there is provided a modified enzyme of SEQ ID NO: 3 comprising at least one amino acid substitution of SEQ ID NO: 3, the at least one amino acid substitution selected from the group consisting of A8E, S114N, N134Y, T136D, F173Y, Y187N, G193K, Q197H, Q197Y, G228D, S267L, A7R, L113Y, Q150F, Q150H, G155A, S171F, V175F, G177C, T199V, F216Y, S223Y, F261Y, and any combinations thereof. The modified enzymes described herein thus each have a sequence with SEQ ID NO: 3 being the base (or original) sequence with one or more amino acid substitution/s of SEQ ID NO: 3.

In an embodiment, the modified enzyme has at least one amino acid substitution selected from a first set of substitutions consisting of A8E, S114N, N134Y, T136D, F173Y, Y187N, G193K, Q197H, Q197Y, G228D, S267L and any combinations thereof. In an embodiment, the modified enzyme has at least one amino acid substitution selected from a second set of substitutions consisting of A7R, L113Y, Q150F, Q150H, G155A, S171F, V175F, G177C, T199V, F216Y, S223Y, F261Y, and any combinations thereof. In an embodiment, the modified enzyme has at least one amino acid substitution each from the first set of substitutions and from the second set of substitutions. In other words, the modified enzyme would have at least two amino acid substitutions of SEQ ID NO: 3.

Preferably, the at least one amino acid substitution is selected from the group consisting of F173Y, Q197H, S267L, and any combinations thereof.

Preferably, the at least one amino acid substation is selected from the group consisting of A7R, L113Y, V175F, G177C, T199V, F216Y, and any combinations thereof.

Preferably, the at least one amino acid substation is selected from the group consisting of V175F, G177C, F216Y, and any combinations thereof.

Preferably, the modified enzyme comprises at least one amino acid substitution selected from a second group consisting of E190C, V209L, E238C, D262G, and in any combinations thereof, preferably all four substitutions in the second group.

Preferably, the modified enzyme comprises at least one amino acid substitution selected from a fourth group consisting of K5, Q15, R30, Q44, K72, S88, A89, T91, K226, S227, G227, T229, S230, N231, D245, and any combinations thereof, preferably Q44L, K72G, K226V, D243Y, and any combinations thereof.

Preferably, the modified enzyme has at least 85% sequence similarity to SEQ ID NO: 3, preferably at least 90% sequence similarity to SEQ ID NO: 3, more preferably at least 92% sequence similarity to SEQ ID NO: 3, even more preferably at least 95% sequence similarity to SEQ ID NO: 3. A 95% sequence similarity to SEQ ID NO: 3 would translate to 13 amino acid substitutions.

In an embodiment, the modified enzyme has a sequence selected from the group consisting of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 65, SEQ ID NO: 66, SEQ ID NO: 67, SEQ ID NO: 68, SEQ ID NO: 69. Preferably, the sequence is selected from the group consisting of SEQ ID NO: 15, SEQ ID NO: 28, SEQ ID NO: 68, and SEQ ID NO: 69.

In a second aspect, there is provided a method of forming a first triglyceride, the method comprises mixing a second triglyceride, a first fatty acid, and the modified enzyme according to the first aspect; and forming the first triglyceride under suitable conditions, wherein the first triglyceride is a mixture of fatty acids from the second triglyceride and the first fatty acid.

Preferably, the first fatty acid is regioselectively attached to the 1 and/or 3 position of the second triglyceride to form the first triglyceride.

Preferably, the second triglyceride has identical fatty acids. Examples include tripalmitin and triolein.

Preferably, the suitable conditions comprises a temperature of at least 55° C., preferably 60° C., and more preferably 70° C.

In an embodiment, the first triglyceride is 1,3-dioleoyl-2-palmitoylglycerol (OPO), 1-oleoyl-2-palmitoyl-3-linoleoylglycerol (OPL), or 1,3-stearoyl-2-oleoyl-glycerol (SOS).

In an embodiment, the first triglyceride is 1,3-dioleoyl-2-palmitoylglycerol (OPO), the second triglyceride is tripalmitin, and the first fatty acid is oleic acid.

In an embodiment, the first triglyceride is 1,3-stearoyl-2-oleoyl-glycerol (SOS), the second triglyceride is triolein, and the fatty acid is stearic acid. In an embodiment, triolein is sourced from high oleic sunflower oil.

In an embodiment, the method comprises mixing a second fatty acid with the second triglyceride, the first fatty acid, and the modified enzyme, and the first triglyceride is a mixture of fatty acids from the second triglyceride, the first fatty acid and the second fatty acid, wherein the first fatty acid is different from the second fatty acid. In an embodiment, the first triglyceride is 1-oleoyl-2-palmitoyl-3-linoleoylglycerol (OPL), the second triglyceride is tripalmitin, the first fatty acid is oleic acid, and the second fatty acid is linoleic acid. Preferably, a weight ratio of the first fatty acid to the second fatty acid is from 1:9 to 9:1.

Preferably, tripalmitin is sourced from a fractionated fraction of palm oil enriched in tripalmitin.

In a third aspect, there is provided a method of producing biodiesel, the method comprises mixing a fatty acid or a triglyceride, a short-chained alcohol, and the modified enzyme according to the first aspect under suitable conditions to form a fatty acid ester of the short-chained alcohol.

Preferably, the short-chained alcohol is selected from the group consisting of methanol, ethanol, n-propanol and isopropanol.

Preferably, the fatty acid or the triglyceride is one or more selected from crude palm oil, sludge oil, fatty matter, used or unused cooking oil, palm fatty acid distillate, tallow, and brown grease.

Advantageously, the modified enzymes described herein may be used as lipases, especially in reactions with higher reaction temperatures. The tolerance of higher reaction temperatures allow the enzyme to be used in more reactions and may be particularly useful in reactions requiring high reaction temperatures. For example, a higher reaction temperature may be needed to melt the substrate. For example, the modified enzymes may be used in the interesterification of triglycerides and the production of biodiesel (such as fatty acid methyl esters).

### DESCRIPTION OF FIGURES (FIGS.)

FIG. 1A and FIG. 1B show the Weblogo of ROL with positions of ROL targeted for mutagenesis highlighted.

FIG. 2 shows the list of ROL single mutants tested during the first and second round of screening.

FIG. 3 shows the relative activity and fold improvement of the single mutants of ROL-4× measured at 60° C. and 45° C.

FIG. 4 shows the relative activity and fold improvement of the mutants of ROL-4× measured at 65° C. and 45° C.

FIG. 5 shows the relative activity and fold improvement of the single mutants of ROL-7× measured at 67° C. and 45° C.

FIG. 6 shows the relative activity and fold improvement of the mutants of ROL-7× measured at 70° C. and 45° C.

FIG. 7 shows the SDS-PAGE analysis of the ROL mutants expressed from Pichia pastoris. Lane 1 is the control, Lanes 2 and 3 are ROL-WT, Lanes 4 and 5 are ROL-4×, Lanes 6 and 7 are ROL-7×, and Lanes 8 and 9 are ROL-10×. The expected size of ROL are marked with asterisks in the band indicated by the arrow.

FIG. 8 shows the thermostability of ROL mutants by HOSun hydrolysis.

FIG. 9 shows the thermostability of ROL mutants by TLC assay with the various lanes on the TLC assay showing the amount of reactant (tricaprylin C24), intermediate (C34), and product (C44) formed at different reaction temperatures. The EIE reaction was carried out using tricaprylin and oleic acid as substrates.

FIG. 10 shows the production of biodiesel and methanol tolerance of ROL mutants by TLC assay. Panel A shows the biodiesel production with HOSun and 50% methanol for Control in Lane 1, ROL-WT in Lane 2, ROL-4× in Lane 3, ROL-7× in Lane 4, ROL-10× in Lane 5, and Eversa in Lane 6. Panel B shows the methanol tolerance of ROL-7×, ROL-10× and Eversa at different methanol concentrations. FAME refers to fatty acid methyl ester, TAG refers to triacylglyceride, FFA refers to free fatty acid, DAG refers to diacylglyceride, and MAG refers to monoacylglyceride.

FIG. 11 shows a list of residues from ROL targeted for B-factor mutagenesis.

FIG. 12 shows the relative activity of the mutants of ROL-10× measured at 70° C./73° C./75° C. and normalized with activity at 45° C.

FIG. 13 shows the thermostability of ROL-10× mutants by HOSun hydrolysis.

FIG. 14 shows the thermostability of ROL-10× mutants by TLC assay. The EIE reaction was carried out using tricaprylin (C24) and oleic acid as substrates. C34 is the intermediate product with one oleic acid substituted and C44 is the product with two oleic acids substituted as per FIG. 9

### DETAILED DESCRIPTION

In the description herein, numerous specific details are set forth in order to provide a thorough understanding of various illustrative embodiments of the invention. It will be understood, however, to one skilled in the art, that embodiments of the invention may be practiced without some or all of these specific details. Embodiments described in the context of one of the methods or devices are analogously valid for the other methods or devices. Similarly, embodiments described in the context of a method are analogously valid for a device, and vice versa.

Unless defined otherwise, all technical and scientific terms used herein have the meaning commonly understood by a person skilled in the art to which this invention belongs.

As used herein, the articles “a”, “an” and “the” as used with regard to a feature or element include a reference to one or more of the features or elements. As used herein, the term “and/or” includes any and all combinations of one or more of the associated listed items. As used herein, the terms “first,” “second,” and “third,” etc. are used merely as labels, and are not intended to impose numerical requirements on their objects.

Where a combination is disclosed, each subcombination of the elements of that combination is also specifically disclosed and is within the scope of the invention. Conversely, where different elements or groups of elements are disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention. Where a value being discussed has inherent limits, for example where a component can be present at a concentration of from 0 to 100%, or where the pH of an aqueous solution can range from 1 to 14, those inherent limits are specifically disclosed. Where a value is explicitly recited, it is to be understood that values which are about the same quantity or amount as the recited value are also within the scope of the invention, as are ranges based thereon.

Although each of these terms has a distinct meaning, the terms “comprising”, “consisting of” and “consisting essentially of” may be interchanged for one another throughout the instant application. The term “having” has the same meaning as “comprising” and may be replaced with either the term “consisting of” or “consisting essentially of”.

The terms “polypeptide” and “protein”, used interchangeably herein, refer to a polymer of amino acids without regard to the length of the polymer; thus, peptides, oligopeptides, and proteins are included within the definition of polypeptide. This term also does not specify or exclude chemical or post-expression modifications of the polypeptides of the invention, although chemical or post-expression modifications of these polypeptides may be included or excluded as specific embodiments. Therefore, for example, modifications to polypeptides that include the covalent attachment of glycosyl groups, acetyl groups, phosphate groups, lipid groups and the like are expressly encompassed by the term polypeptide. Further, polypeptides with these modifications may be specified as individual species to be included or excluded from the present invention. The natural or other chemical modifications, such as those listed in examples above can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. Also included within the definition are polypeptides which contain one or more analogs of an amino acid (including, for example, non-naturally occurring amino acids, amino acids which only occur naturally in an unrelated biological system, modified amino acids from mammalian systems, etc.), polypeptides with substituted linkages, as well as other modifications known in the art, both naturally occurring and non-naturally occurring.

The terms “sequence similarity”, “percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Identity is evaluated using any of the variety of sequence comparison algorithms and programs known in the art. Such algorithms and programs include, but are by no means limited to, TBLASTN, BLASTP, FASTA, TFASTA, CLUSTAL W, FASTDB [Pearson and Lipman, (1988), Proc. Natl. Acad. Sci. USA 85 (8): 2444-2448; Altschul et al., (1990), J. Mol. Biol. 215 (3): 403-410; Thompson et al. (1994), Nucleic Acids Res. 22 (2): 4673-4680; Higgins et al., (1996), Meth. Enzymol. 266:383-402; Altschul et al., (1993), Nature Genetics 3:266-272; Brutlag et al. (1990) Comp. App. Biosci. 6:237-24], the disclosures of which are incorporated by reference in their entireties.

The following detailed description is merely exemplary in nature and is not intended to limit the invention or the application and uses of the invention. Furthermore, there is no intention to be bound by any theory presented in the preceding background of the invention or the following detailed description. Other technical advantages may become readily apparent to one of ordinary skill in the art after review of the following figures and description.

Most of the screening methods described in the literature rely on halo assays or the use of PNP-esters for the rapid identification of lipase mutants with increased thermostability.^{4,6} However, these methods often lead to many false-positive mutants as the assay involves the use of substrates that are fully solubilized in the aqueous layer. Lipases require an aqueous and oil interfacial layer for enzyme activation and hence will work best when the substrate is in excess.⁷ To mimic the oil-rich conditions of an EIE reaction, a spectrophotometric-based screening assay was developed by using 2,3-dimercapto-1-propanol tributyrate (DMPTB) and 5,5-dithio-bis(2-dinitrobenzoic acid) (DTNB).⁸ Hydrolysis of DMPTB will release a free thiol group that can react with DTNB to produce an absorbance reading at 405 nm. Using the information from the sequence alignment of ROL, 26 single mutants of ROL were identified, constructed and screened for increased thermostability with the DMPTB/DTNB assay. All the mutations were constructed based on the quadruple ROL mutant (V209L/D262G/E190C/E238C) described by Zhao et al.⁵ Single mutants with higher thermostability were recombined by gene shuffling and undergo a second round of screening to identify mutants with even higher thermostability. After a few rounds of screening, a variant of ROL containing 6 new mutations (F173Y/V175F/G177C/Q197H/F216Y/S267L) was identified that allows the enzyme to withstand temperatures up to 70° C. It was demonstrated that the enzyme can be used to catalyze the EIE reaction at higher temperatures than before.

Sequence Alignment of ROL and Visualization with WebLogo

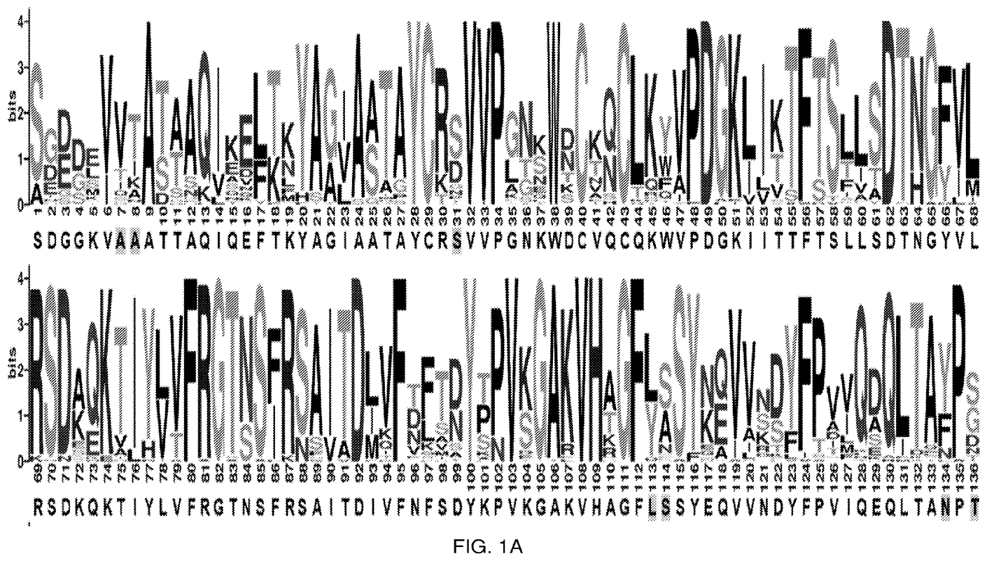

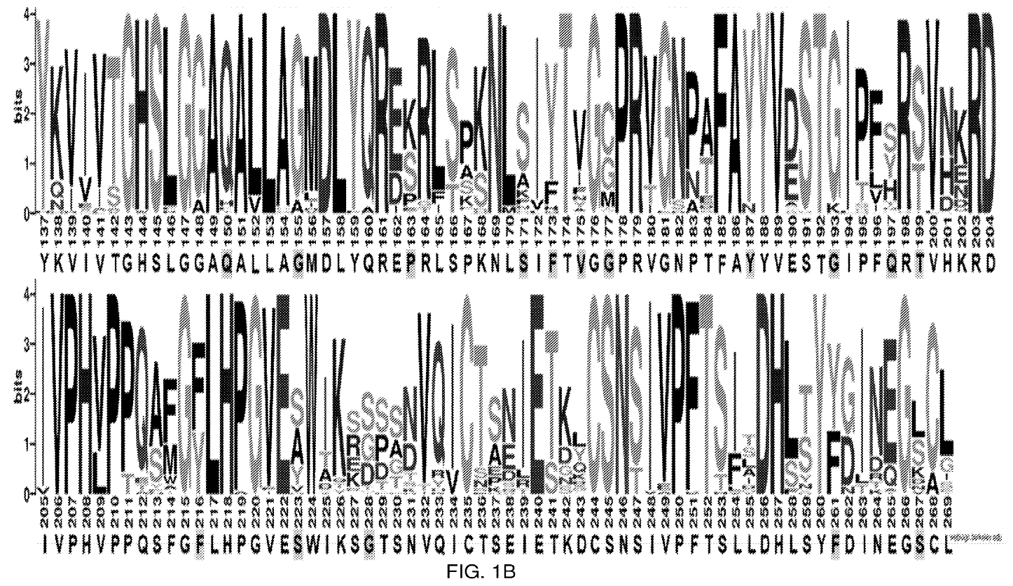

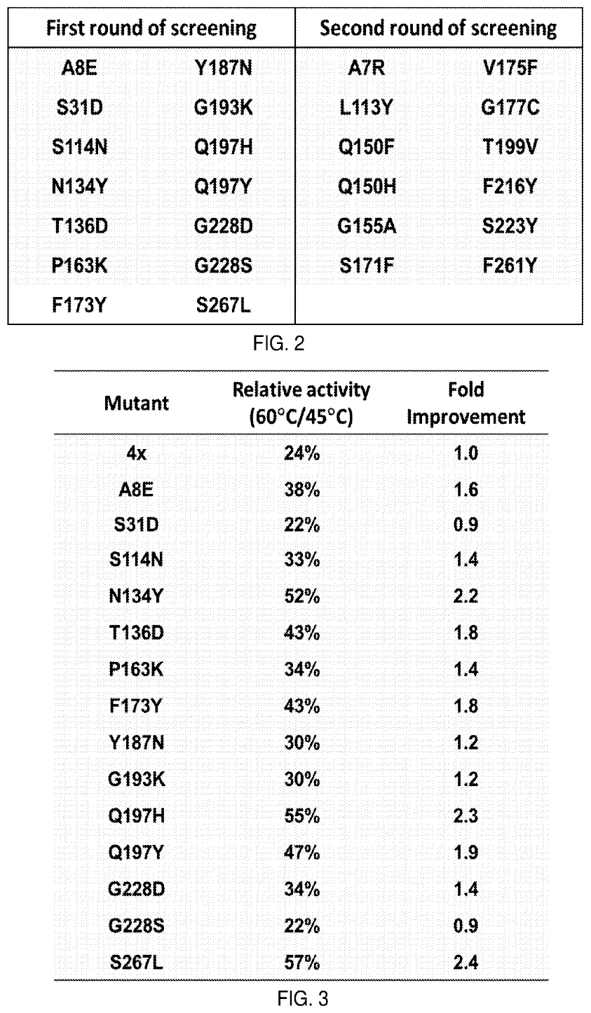

The sequence of our wild-type ROL (SEQ ID NO: 1) shares a 97.8% sequence identity with P61871 (Uniprot). A sequence alignment of ROL-WT was created from the top 50 orthologous sequences of ROL identified from BLAST and visualized using WebLogo (FIG. 1 shown with the mature peptide of ROL, SEQ ID NO: 2).⁹ The WebLogo of ROL provides useful information on the degree of conservation and natural abundance for each residue. A total of 14 and 12 residues were targeted for mutagenesis in the first and second round of screening respectively (FIG. 2). These mutants were created based on their ability to form thermo-stabilizing interactions within the three-dimensional structure of the protein or according to their natural abundance identified from the WebLogo. The mutants for the first round of screening were synthesised based on the ROL-4× (E190C/V209L/E238C/D262G) mutant since it has a higher thermostability than wild-type ROL.⁵

Expression and Screening of ROL Mutants for Increased Thermostability with DTNB Assay

The vector used for the expression of the ROL mutants has a BleoR gene to confer resistance to Zeocin for plasmid selection in E. coli and P. pastoris. It also possesses a GAP promoter and α-factor secretion tag for constitutive expression and secretion of the lipase protein in P. pastoris. This allows the rapid production and screening of the ROL lipases activity from the culture supernatant, without the need to undergo cell lysis and protein purification. The vector is also compatible to Golden-Gate cloning so various ROL mutants can be constructed and cloned into the vector easily.

To compare the thermostability of each ROL mutant, we inoculated the cells harbouring the mutants into BMDY medium to induce expression and secretion of the protein. The supernatants were harvested and subjected to heat inactivation for 1 h at various temperature. For the first round of screening, the heat inactivation was carried out at 45° C. and 60° C. After the heat treatment, we used the DTNB assay to measure the residual activity of each sample. The residual activity measured at 60° C. was normalized against the activity at 45° C. (FIG. 3). The fold improvement for each mutant was determined by comparison with the 4× mutant and results were summarized in FIG. 3. The N134Y, T136D, F173Y, Q197H, Q197Y and S267L mutants were found to possess a higher thermostability than ROL-4× by at least 1.8-fold.

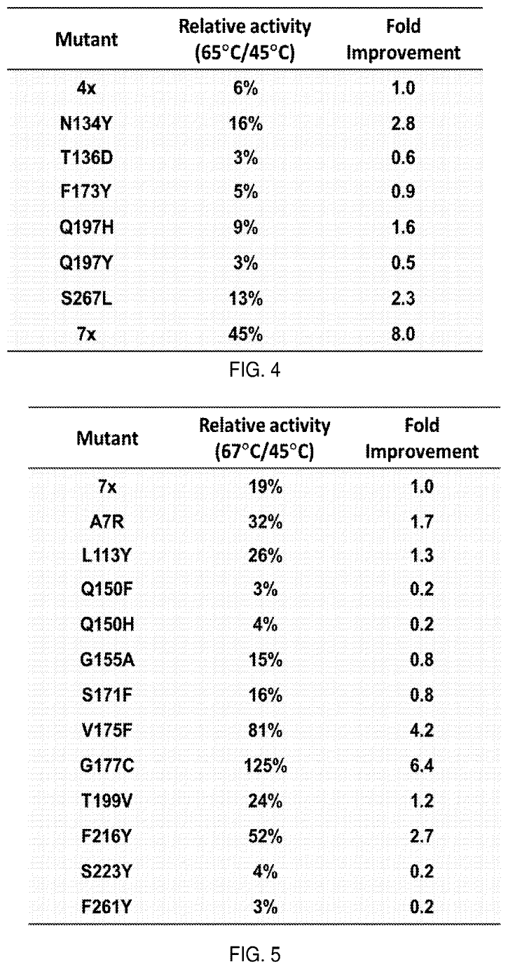

Since the best single mutants only showed slight improvement in thermostability when compared to ROL-4× (1.8 to 2.4-fold), gene shuffling was used to recombine the six mutations identified from the first round of screening into ROL-4×. This allowed rapid identification of synergistic mutations that can lead to a further improvement in the thermostability of ROL. After screening around 500 mutants, ROL-7× (with 3 additional mutations F173Y/Q197H/S267L) was identified with a much higher thermostability than ROL-4× and its single mutants (FIG. 4). ROL-7× retains about 45% of its activity at 65° C. and has an 8-fold increase in thermostability over the 4× mutant.

Second Round of Screening with ROL-7× for Increased Thermostability

Although ROL-7× has increased thermostability over ROL-4×, the activity of ROL-7× drops significantly at temperatures higher than 65° C. To further improve the thermostability of the 7× mutant, an additional 12 residues for mutagenesis were identified (FIGS. 2 and 5). It is postulated that each of these 12 residues may possibly improve the thermostability and enzymatic activity over ROL-4× itself without the additional mutations in ROL-7×. These mutants were synthesised from the 7× template and subjected to the same pipeline that was used during the first round of screening. To identify better mutants, the heat inactivation was increased to 67° C. and 45° C. At 67° C., the ROL-7× mutant retains about 19% of its activity. From the screen, 3 mutants were identified with at least 2-fold increase in thermostability over the 7× mutant (FIG. 5). The V175F, G177C and F216Y mutants have a 4.2, 6.4 and 2.7-fold increase in thermostability over the ROL-7×, respectively.

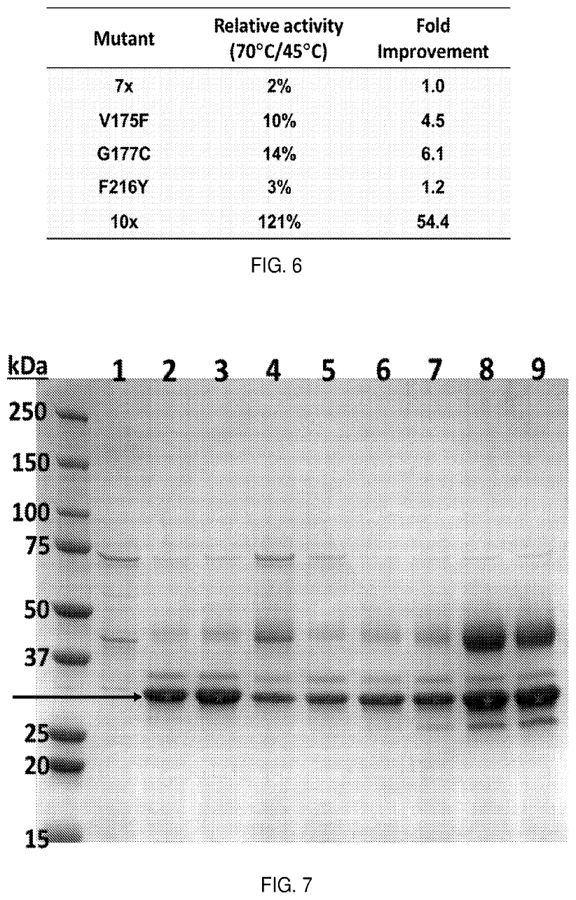

Gene shuffling was used to recombine the mutations found in the single mutants. After screening around 100 mutants, ROL-10× (with 3 additional mutations V175F/G177C/F216Y) was identified with a higher thermostability over ROL-7×. The ROL-10× mutant has a much higher activity at 70° C. than 45° C. (121% increase in activity) and showed a 54-fold improvement in thermostability when compared to ROL-7× (FIG. 6). Other possible mutants that may be synthesised include mutants having one substitution selected from each of the mutants identified in the first and second round of screening (FIG. 2).

### Large Scale Expression of ROL Mutants and Thermostability Assays

To confirm that the ROL mutants identified from the screen have increased thermostability and are able to catalyse the EIE reaction at higher temperatures, the three mutants (4×, 7×, 10×) and wild-type ROL were subcloned into a vector containing the AOX1 promoter. This allows a higher yield of protein to be produced for the EIE reaction. The enzymes were expressed in P. pastoris using BMMY medium. The supernatants were harvested and concentrated 20-fold for the assays. SDS-PAGE analysis of the concentrated supernatant indicated that the mature ROL is the dominant form of the protein (32 kDa) after it has been secreted into the supernatant (FIG. 7). In the SDS-PAGE analysis shown in FIG. 7, lane 1 contains the control, lanes 2 and 3 contain the ROL-WT lipase, lanes 4 and 5 contain the ROL-4× lipase, lanes 6 and 7 contain the ROL-7× lipase and lanes 8 and 9 contain the ROL-10× lipase. A larger protein fragment (47 kDa) identified for ROL-10× is most likely ROL with the pro-domain that has not been cleaved off from the mature ROL. This might be due to a higher yield of ROL-10× expressed from P. pastoris when compared to the other mutants and the endogenous KEX2 protease from P. pastoris is not able to process all the protein. Protein quantitation was carried out using Bradford assay and the yield of ROL-WT, ROL-4×, ROL-7× and ROL-10× were estimated to be 3 mg, 2 mg, 2 mg and 5 mg from 30 mL of BMMY medium, respectively.

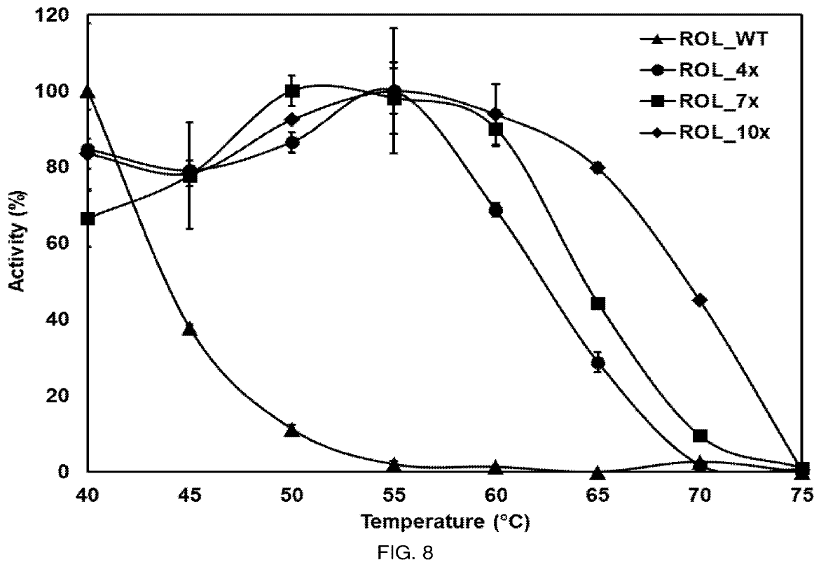

The specific activity of each ROL mutant was measured by titration against the hydrolysed products of HOSun (High Oleic Sunflower oil). At 40° C., the specific activity of ROL-WT, ROL-4×, ROL-7× and ROL-10× were determined to be 900, 2900, 2900 and 4000 U/mg, respectively. To compare the thermostability of the mutants, the reaction was carried out from 40° C.-75° C. in 5° C. intervals and the specific activities of the enzymes were measured (FIG. 8). As may be seen in FIG. 8, ROL-10× was found to have the highest thermostability, followed by ROL-7×, ROL-4× and ROL-WT. This result is consistent with the thermostability assay carried out with DTNB during the screen.

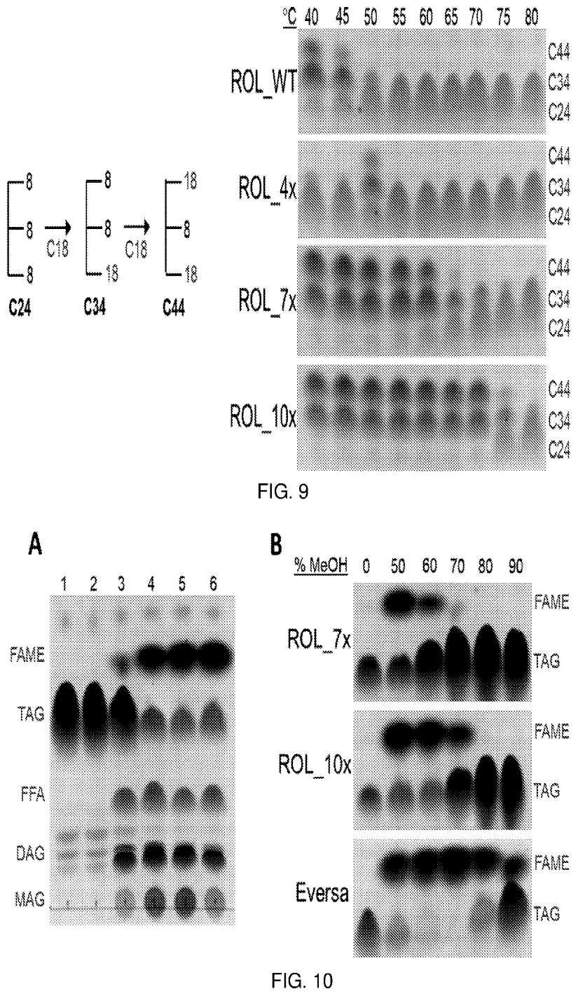

The ROL mutants were used to catalyse the EIE reaction at higher temperatures with tricaprylin (C24) and oleic acid (C18) as substrates for the reaction. Use of tricaprylin is advantageous since the compound does not give a strong background signal on the TLC plate after staining with iodine. Incorporation of one or two molecules of oleic acid into tricaprylin results in the formation of the C34 or C44 products, respectively (FIG. 9). The products can be stained with iodine easily and are able to separate from one another on the TLC plate. This allows for the ease of monitoring whether the EIE reaction has occurred. The reaction was carried out with ROL-WT and the mutants at a temperature range of 40° C.-80° C. The products were separated on the TLC plate for analysis (FIG. 9). The results from the TLC plate suggest that ROL-10× has the highest thermostability and can catalyse the EIE reaction up to 70° C. where significant amounts of the C44 (disubstituted oleate) product is observed up to 70° C. This mutant is thus suitable for use in the industrial production of OPO, SOS and OPL with reaction conditions that can reach up to 60° C.-70° C. The ROL-4× and ROL-7× mutants are able to catalyse the EIE reaction up to 50° C. and 60° C. respectively, which may still be suitable for reactions requiring a lower reaction temperature.

The ROL mutants may be used to prepare a product triglyceride (glycerol esterified with three fatty acids) by the ROL mutants catalysed EIE reaction between a starting triglyceride and one or two fatty acids under suitable conditions, for example a sufficiently high temperature. The ROL mutants allow for selective substitution of the starting triglyceride with the fatty acids to produce a product triglyceride with different fatty acids, for example OPO, SOS, and OPL. In particular, the ROL mutants allow for cheap and readily available triglycerides to be converted to differently substituted triglycerides of higher value.

In an example, OPO may be produced by reacting tripalmitin and oleic acid catalysed by the ROL mutants described herein at a suitable temperature, for example at least 60° C. or 70° C., preferably between 60° C. to 70° C.

In an example, OPL may be produced by reacting tripalmitin, oleic acid and linoleic acid (C18:2) catalysed by the ROL mutants described herein at a suitable temperature, for example at least 60° C. or 70° C., preferably between 60° C. to 70° C.

In an example, SOS may be produced by reacting triolein and stearic acid catalysed by the ROL mutants described herein at a suitable temperature, for example at least 60° C. or 70° C., preferably between 60° C. to 70° C.

A source of tripalmitin may be fractionated palm oil enriched in tripalmitin. A source of triolein may be high oleic sunflower oil (HOSun). Other sources of tripalmitin and triolein may also be used.

### Methanol Tolerance Assay and Production of Biodiesel

Since the thermostability of enzymes and their tolerance towards organic solvents are often well-correlated, the tolerance of ROL mutants towards high concentrations of methanol when compared to ROL-WT were determined. Tolerance of lipases towards methanol allow the lipase to have the potential to be used for the industrial production of biodiesel.

The commercial lipase Eversa can tolerate up to 80% methanol without any significant loss of activity. The concentration of methanol used in the examples herein is stated as volume per volume (% v/v). Production of biodiesel by ROL-WT, ROL-4×, ROL-7×, ROL-10× and Eversa was tested using HOSun and 50% methanol as substrates. After product separation on the TLC plate, it was observed that only ROL-7×, ROL-10× and Eversa produce significant amount of the fatty acid methyl ester (FAME which is biodiesel). To compare the methanol tolerance of ROL-7×, ROL-10× and Eversa, the concentration of methanol was adjusted to between 50-90% for the reaction. ROL-7× was found to be able to retain its activity up to 50% methanol while ROL-10× can retain its activity up to 60% methanol. Although, the methanol tolerance of both ROL mutants is lower than that of Eversa (80%), a strong correlation was observed between the thermostability and methanol tolerance of the ROL mutants.

This suggests that further improvement in the thermostability of ROL may lead to mutants with higher methanol tolerance, which may be used for the production of biodiesel.

### Further Improvement to the Thermostability of ROL-10× by B-Factor Mutagenesis

B-factor mutagenesis is a strategy commonly used to improve the thermostability of enzymes for industrial applications.^{10,11} This approach relies on the structural information that is available to identity residues with high degrees of thermal motion and flexibility (high B values). These residues can be targeted for site-saturation mutagenesis to reduce the rigidity of the enzyme and increase in its thermostability.

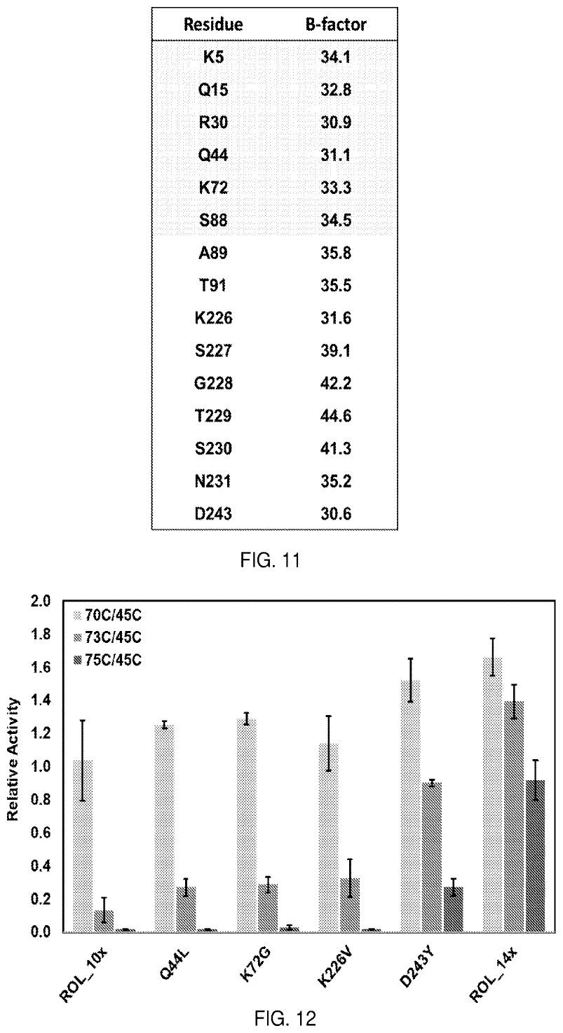

Although ROL-10× can already be used to produce OPO, SOS and OPL, further improvement to the thermostability of the enzyme can help improve its shelf-life and increase yield of the products. Hence, to further improve the thermostability of ROL-10×, 15 residues with the highest B-factor (FIG. 11) were identified from the structure of ROL (PDB: 1LGY) for site-saturation mutagenesis. Each library was constructed from the ROL-10× template using degenerative primers that encode for “NNS” at each of the targeted position. The library size of each mutant library is around 200 clones. The libraries were then expressed in P. pastoris and screened with the DTNB assay described earlier. To identify mutants of ROL-10× with increased thermostability, the activity of each mutant was measured at 73° C. and normalized against the activity at 45° C. Four single mutants (Q44L, K72G, K226V and D243Y) were identified from the screen with increased thermostability over ROL-10× (FIG. 12). The Q44L, K72G, K226V and D243Y mutants have a 2.1, 2.2, 2.5 and 6.8-fold improvement in thermostability at 73° C. over ROL-10×, respectively. Since the improvement in thermostability of the single mutants is only marginal, we recombined the mutations to further improve the thermostability of ROL. ROL-14× contains all the four additional mutations and was found to have a 10.6-fold improvement in activity at 73° C. (FIG. 12).

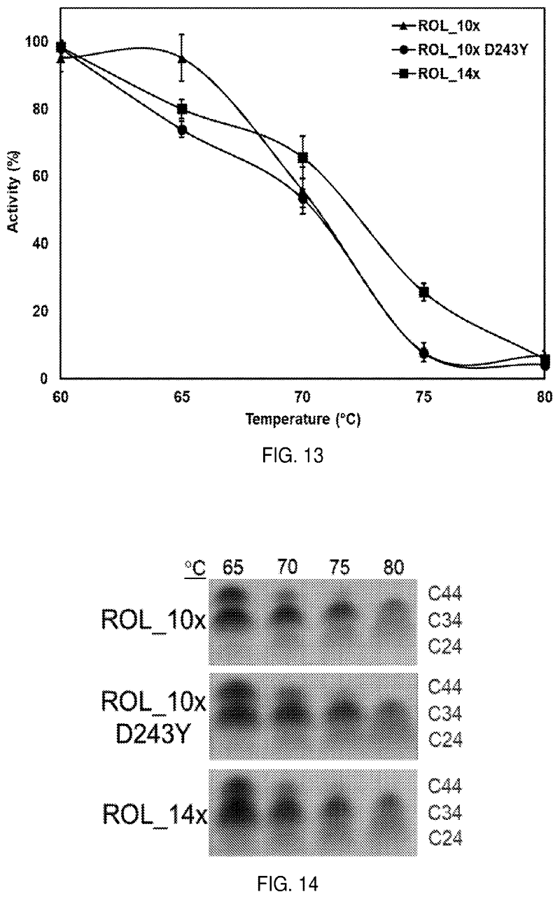

To determine if the increased thermostability of ROL-10× D243Y and ROL-14× mutants can lead to improved yield for the EIE reaction, the two mutants were scaled up for expression in 30 mL BMMY medium. The activities of the concentrated proteins were measured by titration with HOSun at temperatures ranging from 60-80° C. (FIG. 13). Although no improvement in activity was observed with the ROL-10× D243Y mutant across the various temperatures tested, ROL-14× showed significant increase in activity at 75° C. when compared to ROL-10×. At 75° C., ROL-10× was only able to retain 8% of its activity while ROL-14× could retain 26% of its activity. The EIE reaction was subsequently carried as described earlier, with temperature ranging from 65° C.-80° C. No significant improvement in activity was observed with ROL-10× D243Y and ROL-14× compared to ROL-10× (FIG. 14). This suggests that the improvement in thermostability observed for ROL-14× may be limited to hydrolysis and may not have a significant effect for the EIE reaction under the test conditions.

Expression of ROL in P. pastoris

For the small scale expression of ROL in P. pastoris using 96-deepwell plates, cells were first inoculated into 900 μL of BMDY medium containing 1% w/v yeast extract, 2% w/v peptone, 100 mM potassium phosphate, pH 6.0, 1.34% w/v yeast nitrogen base (Formedium), 2% w/v glucose, 0.00004% w/v biotin (Sigma) and 100 μg/mL Zeocin (InvivoGen). The cells were then incubated at 30° C. on a microplate shaker with shaking at 1500 rpm. After 3 days, the cells were pelleted by centrifugation (3000 g for 5 min) and the supernatant can be used directly for the lipase assay.

To obtain the ROL protein with higher yield and concentration, expression of the ROL gene is driven by Aox1 (methanol-inducible) promoter instead of the GAP (constitutive) promoter. Similar to the constitutive expression of ROL, cells were first inoculated into 30 mL of BMGY medium (replace glucose in BMDY with 1% v/v glycerol) and grown overnight at 30° C. for the cells to accumulate a high cell density. The cells were then harvested and diluted to a concentration of 6 OD in BMMY medium (replace glucose in BMDY with 1% v/v methanol). The cells were then incubated at 30° C. with shaking for an additional 3 days. An additional 1% v/v methanol was added to the cells twice on the first and second day of incubation. After 3 days, the supernatant can be harvested by centrifugation. The supernatant was then concentrated 20-fold to 1.5 mL using centrifugal filter units with a 10 kDa cutoff filter (Amicron).

### Lipase Assay Using DTNB

To screen for ROL mutants with higher thermostability, 50 μL of the supernatant containing ROL was first subjected to heat-inactivation for 1 hour at various temperature. To set up the reaction, the following components were transferred to a 2 mL Eppendorf tube: 9.875 μL of C8/C10 methyl esters (Wilmar), 0.125 μL of DMPTB (Sigma), 68 μL of Tris (100 mM, pH 8.0), 2 μL of DTNB (Sigma) (50 mM in DMSO) and 20 μL of supernatant. The reaction was incubated on a thermomixer at various temperature with shaking at 2000 rpm for 1 hour. The reaction was stopped by adding 125 L of acetonitrile and 25 μL of Tris (2M, pH 8.0). The absorbance of the reaction mixture was recorded using a microplate reader at 405 nm.

### Gene Shuffling by PCR and Golden-Gate Cloning

The single mutants of ROL-4× identified from the first round of screening were combined by gene shuffling using Q5 DNA polymerase (NEB) for PCR. The following sets of templates and primers (Table 1) were used to generate three partial fragments of ROL that can be assembled by Golden Gate cloning using Bsal (NEB) and T4 DNA ligase (NEB).

TABLE 1
Sets of templates and primers to generate
partial fragments of ROL in the first
round of screening

| Fragment | Template | Primer |
|---|---|---|
| 1 | ROL-4x | For: CTGGTCTCGGATGGTGCCCGTTAG |
| | | CGGAAAG (SEQ ID NO: 29) |
| | | Rev: CTGGTCTCGTGTAGGTTGGGTTAG |
| | | CAGTCAACTGCTC (SEQ ID NO: 30) |
| 1 | ROL- | For: CTGGTCTCGGATGGTGCCCGTTAG |
| | N134Y | CGGAAAG (SEQ ID NO: 31) |
| | | Rev: CTGGTCTCGTGTAGGTTGGATAAG |
| | | CAGTCAACTGCTC (SEQ ID NO: 32) |
| 1 | ROL-4x | For: CTGGTCTCGGATGGTGCCCGTTAG |
| | | CGGAAAG (SEQ ID NO: 33) |
| | | Rev: CTGGTCTCGTGTAATCTGGGTTAG |
| | | CAGTCAACTGCTC (SEQ ID NO: 34) |
| 1 | ROL- | For: CTGGTCTCGGATGGTGCCCGTTAG |
| | N134Y | CGGAAAG (SEQ ID NO: 35) |
| | | Rev: CTGGTCTCGTGTAATCTGGATAAG |
| | | CAGTCAACTGCTC (SEQ ID NO: 36) |
| 2 | ROL-4x | For: CTGGTCTCGTACAAGGTTATCGTT |
| | | ACCGGTCACTCTC (SEQ ID NO: 37) |
| | | Rev: CTGGTCTCGGAATGGGATACCAGT |
| | | GGAGCAAAC (SEQ ID NO: 38) |
| 2 | ROL- | For: CTGGTCTCGTACAAGGTTATCGTT |
| | F173Y | ACCGGTCACTCTC (SEQ ID NO: 39) |
| | | Rev: CTGGTCTCGGAATGGGATACCAGT |
| | | GGAGCAAAC (SEQ ID NO: 40) |
| 3 | ROL-4x | For: CTGGTCTCGATTCCAGAGAACCGT |
| | | TCACAAGAGAGACATC (SEQ ID NO: |
| | | 41) |
| | | Rev: CTGGTCTCGGCTACAAACAGGAAC |
| | | CCTCGTTAATAC (SEQ ID NO: 42) |
| 3 | ROL-4x | For: CTGGTCTCGATTCCATAGAACCGT |
| | | TCACAAGAGAGACATC (SEQ ID NO: |
| | | 43) |
| | | Rev: CTGGTCTCGGCTACAAACAGGAAC |
| | | CCTCGTTAATAC (SEQ ID NO: 44) |
| 3 | ROL-4x | For: CTGGTCTCGATTCTACAGAACCGT |
| | | TCACAAGAGAGACATC (SEQ ID NO: |
| | | 45) |
| | | Rev: CTGGTCTCGGCTACAAACAGGAAC |
| | | CCTCGTTAATAC (SEQ ID NO: 46) |
| 3 | ROL- | For: CTGGTCTCGATTCCAGAGAACCGT |
| | S267L | TCACAAGAGAGACATC (SEQ ID NO: |
| | | 47) |
| | | Rev: CTGGTCTCGGCTACAAACACAAAC |
| | | CCTCGTTAATACCGAAGTAGG (SEQ ID |
| | | NO: 48) |
| 3 | ROL- | For: CTGGTCTCGATTCCATAGAACCGT |
| | S267L | TCACAAGAGAGACATC (SEQ ID NO: |
| | | 49) |
| | | Rev: CTGGTCTCGGCTACAAACACAAAC |
| | | CCTCGTTAATACCGAAGTAGG (SEQ ID |
| | | NO: 50) |
| 3 | ROL- | For: CTGGTCTCGATTCTACAGAACCGT |
| | S267L | TCACAAGAGAGACATC (SEQ ID NO: |
| | | 51) |
| | | Rev: CTGGTCTCGGCTACAAACACAAAC |
| | | CCTCGTTAATACCGAAGTAGG (SEQ ID |
| | | NO: 52) |

For the second round of screening, the following primers and templates (Table 2) were used to amplify two fragments of ROL from the single mutants of ROL-7×.

TABLE 2
Sets of templates and primers to generate
partial fragments of ROL in the second
round of screening

| Fragment | Template | Primer |
|---|---|---|
| 1 | ROL-7x | For: CTGGTCTCGGATGGTTCCAGTTTC |
| | | TGGTAAGTCTG (SEQ ID NO: 53) |
| | | Rev: CTGGTCTCGTTGGACCACCAACGG |
| | | TGTAGATGG (SEQ ID NO: 54) |
| 1 | ROL-7x | For: CTGGTCTCGGATGGTTCCAGTTTC |
| | | TGGTAAGTCTG (SEQ ID NO: 55) |
| | | Rev: CTGGTCTCGTTGGACCACCAAAGG |
| | | TGTAGATGGACAAGTTCTTTGGG (SEQ |
| | | ID NO: 56) |
| 1 | ROL-7x | For: CTGGTCTCGGATGGTTCCAGTTTC |
| | | TGGTAAGTCTG (SEQ ID NO: 57) |
| | | Rev: CTGGTCTCGTTGGACAACCAACGG |
| | | TGTAGATGGACAAGTTCTTTGGG (SEQ |
| | | ID NO: 58) |
| 1 | ROL-7x | For: CTGGTCTCGGATGGTTCCAGTTTC |
| | | TGGTAAGTCTG (SEQ ID NO: 59) |
| | | Rev: CTGGTCTCGTTGGACAACCAAAGG |
| | | TGTAGATGGACAAGTTCTTTGGG (SEQ |
| | | ID NO: 60) |
| 2 | ROL-7x | For: CTGGTCTCGCCAAGAGTCGGTAAC |
| | | CCAACTTTC (SEQ ID NO: 61) |
| | | Rev: CTGGTCTCGGCTACAAACACAAAC |
| | | CCTCGTTAATACCGAAGTAGG (SEQ ID |
| | | NO: 62) |
| 2 | ROL- | For: CTGGTCTCGCCAAGAGTCGGTAAC |
| | F216Y | CCAACTTTC (SEQ ID NO: 63) |
| | | Rev: CTGGTCTCGGCTACAAACACAAAC |
| | | CCTCGTTAATACCGAAGTAGG (SEQ ID |
| | | NO: 64) |

### Determination of the Specific Activity of ROL by Titration

The specific activity of ROL and its mutants were determined by detecting the release of oleic acid from the hydrolysis of HOSun (Wilmar). Each reaction contained 150 μL of HOSun, 180 μL of 50 mM Tris, pH 8 and 20 μL of supernatant. After incubation on a thermomixer at 40° C., 2000 rpm for 15 mins, the reaction was stopped by adding 300 μL ethanol containing 0.1% w/v phenolphthalein. The mixture is then titrated against 50 mM sodium hydroxide (NaOH) until the purple color developed. To ensure initial rates were obtained, the supernatant was diluted so that the volume of NaOH added does not exceeds 1 mL. The specific activity of ROL is calculated based on equation (1).

$$Specific activity ( \frac{U}{mg} ) = \frac{( {V}_{NaOH} Sample - {V}_{NaOH} Blank ) \times [ NaOH ]}{{T}_{Rxn} \times {E}_{vol}} \div [ E ]$$

where V_{NaOH} Sample is the volume of NaOH added for the sample in mL, V_{NaOH} Blank is the volume of NaOH added for the blank in mL, [NaOH] is the concentration of the NaOH used for the titration in mM, T_{R×n} is the duration of the reaction in min, Evol is the enzyme in mL and [E] is the concentration of the enzyme in mg/mL.

To measure the specific activity of ROL mutants at higher temperature, 50 μL of the supernatant was incubated on a thermocycler at the selected temperature for 1 hour. The reaction was then carried out at the temperature used for the inactivation of the enzyme.

### Analysis of EIE Reaction by TLC Assay

The EIE was carried out by setting up the reaction in a 2 mL Eppendorf tube. The reaction contained 25 μL of Tricaprylin (Sigma), 50 μL oleic acid (Wilmar) and 25 μL supernatant (concentrated). The reaction was incubated on a thermomixer set to 40° C.-80° C., 2000 rpm for 1 hour. The reaction was stopped by adding 750 μL isopropanol and 1 μL of the sample was spotted on the Silica gel 60 F254 TLC plate (Merck). The TLC plate was developed using a solvent system containing hexane and diethyl ether (80:20). After 10 min, the plate was air dried and stained with iodine.

### Methanol Tolerance of ROL Mutants

Production of biodiesel was carried out in a 2 mL Eppendorf tube containing 100 μL HOSun and 10 μL supernatant (concentrated). 10-90 μL methanol was also added to achieve a methanol concentration of 50-90%. The reaction was incubated on a thermomixer set to 40° C., 2000 rpm for 24 hours. The reaction was stopped by adding 900 μL isopropanol and 1 μL of the sample was spotted on the TLC plate. The TLC plate was developed using a solvent system containing hexane, diethyl ether and formic acid (80:20:1) for 5 min. The TLC plate was then air dried and stained with iodine for visualization.

TABLE 3
List of ROL mutants

| | | Mutations with respect to the ROL mature peptide |
|---|---|---|
| Mutants | SEQ ID | (SEQ ID NO: 2) |
| ROL-4x | SEQ ID NO: 3 | E190C/V209L/E238C/D262G |
| ROL-A8E | SEQ ID NO: 4 | A8E/E190C/V209L/E238C/D262G |
| ROL-S114N | SEQ ID NO: 5 | S114N/E190C/V209L/E238C/D262G |
| ROL-N134Y | SEQ ID NO: 6 | N134Y/E190C/V209L/E238C/D262G |
| ROL-T136D | SEQ ID NO: 7 | T136D/E190C/V209L/E238C/D262G |
| ROL-F173Y | SEQ ID NO: 8 | F173Y/E190C/V209L/E238C/D262G |
| ROL-Y187N | SEQ ID NO: 9 | Y187N/E190C/V209L/E238C/D262G |
| ROL-G193K | SEQ ID NO: 10 | E190C/G193K/V209L/E238C/D262G |
| ROL-Q197H | SEQ ID NO: 11 | E190C/Q197H/V209L/E238C/D262G |
| ROL-Q197Y | SEQ ID NO: 12 | E190C/Q197Y/V209L/E238C/D262G |
| ROL-G228D | SEQ ID NO: 13 | E190C/V209L/G228D/E238C/D262G |
| ROL-S267L | SEQ ID NO: 14 | E190C/V209L/E238C/D262G/S267L |
| ROL-7x | SEQ ID NO: 15 | F173Y/E190C/Q197H/V209L/E238C/D262G/ |
| | | S267L |
| ROL-A7R | SEQ ID NO: 16 | A7R/F173Y/E190C/Q197H/V209L/E238C/ |
| | | D262G/S267L |
| ROL-L113Y | SEQ ID NO: 17 | L113Y/F173Y/E190C/Q197H/V209L/E238C/ |
| | | D262G/S267L |
| ROL-Q150F | SEQ ID NO: 18 | Q150F/F173Y/E190C/Q197H/V209L/E238C/ |
| | | D262G/S267L |
| ROL-Q150H | SEQ ID NO: 19 | Q150H/F173Y/E190C/Q197H/V209L/E238C/ |
| | | D262G/S267L |
| ROL-G155A | SEQ ID NO: 20 | G155A/F173Y/E190C/Q197H/V209L/E238C/ |
| | | D262G/S267L |
| ROL-S171F | SEQ ID NO: 21 | S171F/F173Y/E190C/Q197H/V209L/E238C/ |
| | | D262G/S267L |
| ROL-V175F | SEQ ID NO: 22 | F173Y/V175F/E190C/Q197H/V209L/E238C/ |
| | | D262G/S267L |
| ROL-G177C | SEQ ID NO: 23 | F173Y/G177C/E190C/Q197H/V209L/E238C/ |
| | | D262G/S267L |
| ROL-T199V | SEQ ID NO: 24 | F173Y/E190C/Q197H/T199V/V209L/E238C/ |
| | | D262G/S267L |
| ROL-F216Y | SEQ ID NO: 25 | F173Y/E190C/Q197H/V209L/F216Y/E238C/ |
| | | D262G/S267L |
| ROL-S223Y | SEQ ID NO: 26 | F173Y/E190C/Q197H/V209L/S223Y/E238C/ |
| | | D262G/S267L |
| ROL-F261Y | SEQ ID NO: 27 | F173Y/E190C/Q197H/V209L/E238C/F261Y/ |
| | | D262G/S267L |
| ROL-10x | SEQ ID NO: 28 | F173Y/V175F/G177C/E190C/Q197H/V209L/ |
| | | F216Y/E238C/D262G/S267L |
| ROL-10x | SEQ ID NO: 65 | Q44L/F173Y/V175F/G177C/E190C/Q197H/ |
| Q44L | | V209L/F216Y/E238C/D262G/S267L |
| ROL-10x | SEQ ID NO: 66 | K72G/F173Y/V175F/G177C/E190C/Q197H/ |
| K72G | | V209L/F216Y/E238C/D262G/S267L |
| ROL-10x | SEQ ID NO: 67 | F173Y/V175F/G177C/E190C/Q197H/V209L/ |
| K226V | | F216Y/K226V/E238C/D262G/S267L |
| ROL-10x | SEQ ID NO: 68 | F173Y/V175F/G177C/E190C/Q197H/V209L/ |
| D243Y | | F216Y/E238C/D243Y/D262G/S267L |
| ROL-14x | SEQ ID NO: 69 | Q44L/K72G/F173Y/V175F/G177C/E190C/ |
| | | Q197H/V209L/F216Y/K226V/E238C/D243Y/ |
| | | D262G/S267L |

The mutations listed above is with respect to the ROL mature peptide (SEQ ID NO: 2). The mutations are listed with the first alphabet indicating the single letter abbreviation of the original amino acid residue in SEQ ID NO: 2, the number indicating amino acid position in SEQ ID NO: 2, and the second alphabet indicating the single letter abbreviation of the new amino acid residue.

The ROL-7× and ROL-10× mutants appear to be suitable for the catalysis of EIE reaction at high temperature. The data suggests that ROL-7× is able to withstand a reaction temperature of up to 60° C. and ROL-10× can withstand up to 70° C.

Since ROL-7× and ROL-10× were found to be able to tolerate up to 50-60% methanol, these ROL mutants have the potential to be used for the production of biodiesel.

Sequences of selected lipases used herein
Sequence of wild-type ROL with
propeptide portion in bold
(SEQ ID NO: 1)
MVPVSGKSGSSTTAVSASDNSALPPLISSRCAPPSNKGSKSDLQAEPYYM
QKNTEWYESHGGNLTSIGKRDDNLVGGMTLDLPSDAPPISLSGSTNSASD
GGKVAAATTAQIQEFTKYAGIAATAYCRSVVPGNKWDCVQCQKWVPDGKI
ITTFTSLLSDTNGYVLRSDKQKTIYLVFRGTNSFRSAITDIVFNFSDYKP
VKGAKVHAGFLSSYEQVVNDYFPVIQEQLTANPTYKVIVTGHSLGGAQAL
LAGMDLYQREPRLSPKNLSIFTVGGPRVGNPTFAYYVESTGIPFQRTVHK
RDIVPHVPPQSFGFLHPGVESWIKSGTSNVQICTSEIETKDCSNSIVPFT
SLLDHLSYFDINEGSCL
Sequence of ROL mature peptide
(SEQ ID NO: 2)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIFTVGGPRVGNPTFAYYVESTGIPFQRTV
HKRDIVPHVPPQSFGFLHPGVESWIKSGTSNVQICTSEIETKDCSNSIVP
FTSLLDHLSYFDINEGSCL
Sequence of ROL-4x (Amino acid
substitutions with respect to
SEQ ID NO: 2 in bold)
(SEQ ID NO: 3)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIFTVGGPRVGNPTFAYYVCSTGIPFQRTV
HKRDIVPHLPPQSFGFLHPGVESWIKSGTSNVQICTSCIETKDCSNSIVP
FTSLLDHLSYFGINEGSCL
Sequence of ROL-A8E (Amino acid
substitutions with respect to
SEQ ID NO: 2 in bold)
(SEQ ID NO: 4)
SDGGKVAEATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIFTVGGPRVGNPTFAYYVCSTGIPFQRTV
HKRDIVPHLPPQSFGFLHPGVESWIKSGTSNVQICTSCIETKDCSNSIVP
FTSLLDHLSYFGINEGSCL
Sequence of ROL-S114N (Amino acid
substitutions with respect to SEQ
ID NO: 2 in bold)
(SEQ ID NO: 5)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLNSYEQVVNDYFPVIQEQLTANPTYKVIVTGHSLGGAQ
ALLAGMDLYQREPRLSPKNLSIFTVGGPRVGNPTFAYYVCSTGIPFQRTV
HKRDIVPHLPPQSFGFLHPGVESWIKSGTSNVQICTSCIETKDCSNSIVP
FTSLLDHLSYFGINEGSCL
Sequence of ROL-N134Y (Amino acid
substitutions with respect to SEQ
ID NO: 2 in bold)
(SEQ ID NO: 6)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTAYPTYKVIVTGHSLGGAQ
ALLAGMDLYQREPRLSPKNLSIFTVGGPRVGNPTFAYYVCSTGIPFQRTV
HKRDIVPHLPPQSFGFLHPGVESWIKSGTSNVQICTSCIETKDCSNSIVP
FTSLLDHLSYFGINEGSCL
Sequence of ROL-T136D (Amino acid
substitutions with respect to SEQ
ID NO: 2 in bold)
(SEQ ID NO: 7)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPDYKVIVTGHSLGGAQ
ALLAGMDLYQREPRLSPKNLSIFTVGGPRVGNPTFAYYVCSTGIPFQRTV
HKRDIVPHLPPQSFGFLHPGVESWIKSGTSNVQICTSCIETKDCSNSIVP
FTSLLDHLSYFGINEGSCL
Sequence of ROL-F173Y (Amino acid
substitutions with respect to SEQ
ID NO: 2 in bold)
(SEQ ID NO: 8)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIYTVGGPRVGNPTFAYYVCSTGIPFQRTV
HKRDIVPHLPPQSFGFLHPGVESWIKSGTSNVQICTSCIETKDCSNSIVP
FTSLLDHLSYFGINEGSCL
Sequence of ROL-Y187N (Amino acid
substitutions with respect to SEQ
ID NO: 2 in bold)
(SEQ ID NO: 9)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIFTVGGPRVGNPTFANYVCSTGIPFQRTV
HKRDIVPHLPPQSFGFLHPGVESWIKSGTSNVQICTSCIETKDCSNSIVP
FTSLLDHLSYFGINEGSCL
Sequence of ROL-G193K (Amino acid
substitutions with respect to SEQ
ID NO: 2 in bold)
(SEQ ID NO: 10)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIFTVGGPRVGNPTFAYYVCSTKIPFQRTV
HKRDIVPHLPPQSFGFLHPGVESWIKSGTSNVQICTSCIETKDCSNSIVP
FTSLLDHLSYFGINEGSCL
Sequence of ROL-Q197H (Amino acid
substitutions with respect to SEQ
ID NO: 2 in bold)
(SEQ ID NO: 11)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIFTVGGPRVGNPTFAYYVCSTGIPFHRTV
HKRDIVPHLPPQSFGFLHPGVESWIKSGTSNVQICTSCIETKDCSNSIVP
FTSLLDHLSYFGINEGSCL
Sequence of ROL-Q197Y (Amino acid
substitutions with respect to SEQ
ID NO: 2 in bold)
(SEQ ID NO: 12)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIFTVGGPRVGNPTFAYYVCSTGIPFYRTV
HKRDIVPHLPPQSFGFLHPGVESWIKSGTSNVQICTSCIETKDCSNSIVP
FTSLLDHLSYFGINEGSCL
Sequence of ROL-G228D (Amino acid
substitutions with respect to SEQ
ID NO: 2 in bold)
(SEQ ID NO: 13)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIFTVGGPRVGNPTFAYYVCSTGIPFQRTV
HKRDIVPHLPPQSFGFLHPGVESWIKSDTSNVQICTSCIETKDCSNSIVP
FTSLLDHLSYFGINEGSCL
Sequence of ROL-S267L (Amino acid
substitutions with respect to SEQ
ID NO: 2 in bold)
(SEQ ID NO: 14)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIFTVGGPRVGNPTFAYYVCSTGIPFQRTV
HKRDIVPHLPPQSFGFLHPGVESWIKSGTSNVQICTSCIETKDCSNSIVP
FTSLLDHLSYFGINEGLCL
Sequence of ROL-7x (Amino acid
substitutions with respect to
SEQ ID NO: 2 in bold)
(SEQ ID NO: 15)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIYTVGGPRVGNPTFAYYVCSTGIPFHRTV
HKRDIVPHLPPQSFGFLHPGVESWIKSGTSNVQICTSCIETKDCSNSIVP
FTSLLDHLSYFGINEGLCL
Sequence of ROL-A7R (Amino acid
substitutions with respect to
SEQ ID NO: 2 in bold)
(SEQ ID NO: 16)
SDGGKVRAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIYTVGGPRVGNPTFAYYVCSTGIPFHRTV
HKRDIVPHLPPQSFGFLHPGVESWIKSGTSNVQICTSCIETKDCSNSIVP
FTSLLDHLSYFGINEGLCL
Sequence of ROL-L113Y (Amino acid
substitutions with respect to SEQ
ID NO: 2 in bold)
(SEQ ID NO: 17)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFYSSYEQVVNDYFPVIQEQLTANPTYKVIVTGHSLGGAQ
ALLAGMDLYQREPRLSPKNLSIYTVGGPRVGNPTFAYYVCSTGIPFHRTV
HKRDIVPHLPPQSFGFLHPGVESWIKSGTSNVQICTSCIETKDCSNSIVP
FTSLLDHLSYFGINEGLCL
Sequence of ROL-Q150F (Amino acid
substitutions with respect to SEQ
ID NO: 2 in bold)
(SEQ ID NO: 18)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAF
ALLAGMDLYQREPRLSPKNLSIYTVGGPRVGNPTFAYYVCSTGIPFHRTV
HKRDIVPHLPPQSFGFLHPGVESWIKSGTSNVQICTSCIETKDCSNSIVP
FTSLLDHLSYFGINEGLCL
Sequence of ROL-Q150H (Amino acid
substitutions with respect to SEQ
ID NO: 2 in bold)
(SEQ ID NO: 19)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAH
ALLAGMDLYQREPRLSPKNLSIYTVGGPRVGNPTFAYYVCSTGIPFHRTV
HKRDIVPHLPPQSFGFLHPGVESWIKSGTSNVQICTSCIETKDCSNSIVP
FTSLLDHLSYFGINEGLCL
Sequence of ROL-G155A (Amino acid
substitutions with respect to SEQ
ID NO: 2 in bold)
(SEQ ID NO: 20)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAAMDLYQREPRLSPKNLSIYTVGGPRVGNPTFAYYVCSTGIPFHRTV
HKRDIVPHLPPQSFGFLHPGVESWIKSGTSNVQICTSCIETKDCSNSIVP
FTSLLDHLSYFGINEGLCL
Sequence of ROL-S171F (Amino acid
substitutions with respect to SEQ
ID NO: 2 in bold)
(SEQ ID NO: 21)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLFIYTVGGPRVGNPTFAYYVCSTGIPFHRTV
HKRDIVPHLPPQSFGFLHPGVESWIKSGTSNVQICTSCIETKDCSNSIVP
FTSLLDHLSYFGINEGLCL
Sequence of ROL-V175F (Amino acid
substitutions with respect to SEQ
ID NO: 2 in bold)
(SEQ ID NO: 22)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIYTFGGPRVGNPTFAYYVCSTGIPFHRTV
HKRDIVPHLPPQSFGFLHPGVESWIKSGTSNVQICTSCIETKDCSNSIVP
FTSLLDHLSYFGINEGLCL
Sequence of ROL-G177C (Amino acid
substitutions with respect to SEQ
ID NO: 2 in bold)
(SEQ ID NO: 23)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIYTVGCPRVGNPTFAYYVCSTGIPFHRTV
HKRDIVPHLPPQSFGFLHPGVESWIKSGTSNVQICTSCIETKDCSNSIVP
FTSLLDHLSYFGINEGLCL
Sequence of ROL-T199V (Amino acid
substitutions with respect to SEQ
ID NO: 2 in bold)
(SEQ ID NO: 24)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIYTVGGPRVGNPTFAYYVCSTGIPFHRVV
HKRDIVPHLPPQSFGFLHPGVESWIKSGTSNVQICTSCIETKDCSNSIVP
FTSLLDHLSYFGINEGLCL
Sequence of ROL-F216Y (Amino acid
substitutions with respect to SEQ
ID NO: 2 in bold)
(SEQ ID NO: 25)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIYTVGGPRVGNPTFAYYVCSTGIPFHRTV
HKRDIVPHLPPQSFGYLHPGVESWIKSGTSNVQICTSCIETKDCSNSIVP
FTSLLDHLSYFGINEGLCL
Sequence of ROL-S223Y (Amino acid
substitutions with respect to SEQ
ID NO: 2 in bold)
(SEQ ID NO: 26)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIYTVGGPRVGNPTFAYYVCSTGIPFHRTV
HKRDIVPHLPPQSFGFLHPGVEYWIKSGTSNVQICTSCIETKDCSNSIVP
FTSLLDHLSYFGINEGLCL
Sequence of ROL-F261Y (Amino acid
substitutions with respect to SEQ
ID NO: 2 in bold)
(SEQ ID NO: 27)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIYTVGGPRVGNPTFAYYVCSTGIPFHRTV
HKRDIVPHLPPQSFGFLHPGVESWIKSGTSNVQICTSCIETKDCSNSIVP
FTSLLDHLSYYGINEGLCL
Sequence of ROL-10x (Amino acid
substitutions with respect to SEQ
ID NO: 2 in bold)
(SEQ ID NO: 28)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIYTFGCPRVGNPTFAYYVCSTGIPFHRTV
HKRDIVPHLPPQSFGYLHPGVESWIKSGTSNVQICTSCIETKDCSNSIVP
FTSLLDHLSYFGINEGLCL
Sequence of ROL-10x Q44L (Amino acid
substitutions with respect to SEQ ID
NO: 2 in bold)
(SEQ ID NO: 65)
SDGGKVAAATTAQIQEFTKYAGIAATAYCRSVVPGNKWDCVQCLKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIYTFGCPRVGNPTFAYYVCSTGIPFHRTV
HKRDIVPHLPPQSFGYLHPGVESWIKSGTSNVQICTSCIETKDCSNSIVP
FTSLLDHLSYFGINEGLCL
Sequence of ROL-10x K72G (Amino acid
substitutions with respect to SEQ ID
NO: 2 in bold)
(SEQ ID NO: 66)
SDGGKVAAATTAQIQEFTKYAGIAATAYCRSVVPGNKWDCVQCQKWVPDG
KIITTFTSLLSDTNGYVLRSDGQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIYTFGCPRVGNPTFAYYVCSTGIPFHRTV
HKRDIVPHLPPQSFGYLHPGVESWIKSGTSNVQICTSCIETKDCSNSIVP
FTSLLDHLSYFGINEGLCL
Sequence of ROL-10x K226V (Amino acid
substitutions with respect to SEQ ID
NO: 2 in bold)
(SEQ ID NO: 67)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIYTFGCPRVGNPTFAYYVCSTGIPFHRTV
HKRDIVPHLPPQSFGYLHPGVESWIVSGTSNVQICTSCIETKDCSNSIVP
FTSLLDHLSYFGINEGLCL
Sequence of ROL-10x D243Y (Amino acid
substitutions with respect to SEQ ID
NO: 2 in bold)
(SEQ ID NO: 68)
SDGGKVAAATTAQIQEFTKYAGIAATAYCRSVVPGNKWDCVQCQKWVPDG
KIITTFTSLLSDTNGYVLRSDKQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIYTFGCPRVGNPTFAYYVCSTGIPFHRTV
HKRDIVPHLPPQSFGYLHPGVESWIKSGTSNVQICTSCIETKYCSNSIVP
FTSLLDHLSYFGINEGLCL
Sequence of ROL-14x (Amino acid
substitutions with respect to SEQ
ID NO: 2 in bold)
(SEQ ID NO: 69)
SDGGKVAAATTAQIQEFTKYAGIAATAYCRSVVPGNKWDCVQCLKWVPDG
KIITTFTSLLSDTNGYVLRSDGQKTIYLVFRGTNSFRSAITDIVFNFSDY
KPVKGAKVHAGFLSSYEQVVNDYFPVIQEQLTANPTYKVIVTGHSLGGAQ
ALLAGMDLYQREPRLSPKNLSIYTFGCPRVGNPTFAYYVCSTGIPFHRTV
HKRDIVPHLPPQSFGYLHPGVESWIVSGTSNVQICTSCIETKYCSNSIVP
FTSLLDHLSYFGINEGLCL

### REFERENCES

(1) Ghide, M. K.; Yan, Y. 1,3-Dioleoyl-2-palmitoyl glycerol (OPO)-Enzymatic synthesis and use as an important supplement in infant formulas. J Food Biochem 2021, 45 (7), e13799. DOI: 10.1111/jfbc.13799.
(2) Ghazani, S. M.; Marangoni, A. G. Facile lipase-catalyzed synthesis of a chocolate fat mimetic. Sci Rep 2018, 8 (1), 15271. DOI: 10.1038/s41598-018-33600-x.
(3) López-Fernández, J.; Benaiges, M. D.; Valero, F. Rhizopus oryzae Lipase, a Promising Industrial Enzyme: Biochemical Characteristics, Production and Biocatalytic Applications. Catalysts 2020, 10 (11). DOI: 10.3390/catal10111277.
(4) Kohno, M.; Enatsu, M.; Funatsu, J.; Yoshiizumi, M.; Kugimiya, W. Improvement of the optimum temperature of lipase activity for Rhizopus niveus by random mutagenesis and its structural interpretation. J Biotechnol 2001, 87 (3), 203-210. DOI: 10.1016/s0168-1656 (01) 00243-7.
(5) Zhao, J. f.; Wang, Z.; Gao, F. I.; Lin, J. p.; Yang, L. r.; Wu, M. b. Enhancing the thermostability of Rhizopus oryzae lipase by combined mutation of hot-spots and engineering a disulfide bond. RSC Advances 2018, 8 (72), 41247-41254, 10.1039/C8RA07767C. DOI: 10.1039/C8RA07767C.
(6) Li, G.; Fang, X.; Su, F.; Chen, Y.; Xu, L.; Yan, Y. Enhancing the Thermostability of Rhizomucor miehei Lipase with a Limited Screening Library by Rational-Design Point Mutations and Disulfide Bonds. Appl Environ Microbiol 2018, 84 (2). DOI: 10.1128/aem.02129-17.
(7) Reis, P.; Holmberg, K.; Watzke, H.; Leser, M. E.; Miller, R. Lipases at interfaces: a review. Adv Colloid Interface Sci 2009, 147-148, 237-250. DOI: 10.1016/j.cis.2008.06.001.
(8) Farias, R. N.; Torres, M.; Canela, R. Spectrophotometric determination of the positional specificity of nonspecific and 1,3-specific lipases. Anal Biochem 1997, 252 (1), 186-189. DOI: 10.1006/abio.1997.2240.
(9) Crooks, G. E.; Hon, G.; Chandonia, J. M.; Brenner, S. E. WebLogo: a sequence logo generator. Genome Res 2004, 14 (6), 1188-1190. DOI: 10.1101/gr.849004.
(10) Reetz, M. T.; Carballeira, J. D.; Vogel, A.; Iterative saturation mutagenesis on the basis of B factors as a strategy for increasing protein thermostability. Angew Chem Int Ed Engl 2006, 45 (46), 7745-7751. DOI: 10.1002/anie.200602795.
(11) Ge, L.; Li, D.; Wu, T.; Zhao, L.; Ding, G.; Wang, Z.; Xiao, W.; B-factor-saturation mutagenesis as a strategy to increase the thermostability of α-L-rhamnosidase from Aspergillus terreus. J Biotechnol 2018, 275, 17-23. DOI: 10.1016/j.jbiotec.2018.03.013.

1. A modified enzyme of SEQ ID NO: 3 comprising at least one amino acid substitution of SEQ ID NO: 3, the at least one amino acid substitution selected from the group consisting of A8E, S114N, N134Y, T136D, F173Y, Y187N, G193K, Q197H, Q197Y, G228D, S267L, A7R, L113Y, Q150F, Q150H, G155A, S171F, V175F, G177C, T199V, F216Y, S223Y, F261Y, and any combinations thereof.

2. The modified enzyme according to claim 1, wherein the at least one amino acid substitution is selected from the group consisting of F173Y, Q197H, S267L, and any combinations thereof.

3. The modified enzyme according to claim 1 or 2, wherein the at least one amino acid substation is selected from the group consisting of A7R, L113Y, V175F, G177C, T199V, F216Y, and any combinations thereof.

4. The modified enzyme according to claim 3, wherein the wherein the at least one amino acid substation is selected from the group consisting of V175F, G177C, F216Y, and any combinations thereof.

5. The modified enzyme according to any one of claims 1 to 4 comprising at least one amino acid substitution selected from a second group consisting of E190C, V209L, E238C, D262G, and in any combinations thereof, preferably all four substitutions in the second group.

6. The modified enzyme according to any one of claims 1 to 5, comprising at least one amino acid substitution selected from a fourth group consisting of K5, Q15, R30, Q44, K72, S88, A89, T91, K226, S227, G227, T229, S230, N231, D245, and any combinations thereof, preferably Q44L, K72G, K226V, D243Y, and any combinations thereof.

7. The modified enzyme according to any one of claims 1 to 6, wherein the modified enzyme has at least 85% sequence similarity to SEQ ID NO: 3, preferably at least 90% sequence similarity to SEQ ID NO: 3, more preferably at least 92% sequence similarity to SEQ ID NO: 3, even more preferably at least 95% sequence similarity to SEQ ID NO: 3.

8. The modified enzyme according to claim 1 with a sequence selected from the group consisting of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 65, SEQ ID NO: 66, SEQ ID NO: 67, SEQ ID NO: 68, SEQ ID NO: 69.

9. The modified enzyme according to claim 8, wherein the sequence is selected from the group consisting of SEQ ID NO: 15, SEQ ID NO: 28, SEQ ID NO: 68, and SEQ ID NO: 69.

10. A method of forming a first triglyceride, the method comprises mixing a second triglyceride, a first fatty acid, and the modified enzyme according to any one of claims 1 to 9; and forming the first triglyceride under suitable conditions, wherein the first triglyceride is a mixture of fatty acids from the second triglyceride and the first fatty acid.

11. The method according to claim 10, wherein the first fatty acid is regioselectively attached to the 1 and/or 3 position of the second triglyceride to form the first triglyceride.

12. The method according to claim 10 or 11, wherein the second triglyceride has identical fatty acids.

13. The method according to any one of claims 10 to 12, wherein the suitable conditions comprises a temperature of at least 55° C., preferably 60° C., and more preferably 70° C.

14. The method according to any one of claims 10 to 13, wherein the first triglyceride is 1,3-dioleoyl-2-palmitoylglycerol (OPO), 1-oleoyl-2-palmitoyl-3-linoleoylglycerol (OPL), or 1,3-stearoyl-2-oleoyl-glycerol (SOS).

15. The method according to claim 14, wherein the first triglyceride is 1,3-dioleoyl-2-palmitoylglycerol (OPO), the second triglyceride is tripalmitin, and the first fatty acid is oleic acid.

16. The method according to claim 14, wherein the first triglyceride is 1,3-stearoyl-2-oleoyl-glycerol (SOS), the second triglyceride is triolein, and the fatty acid is stearic acid.

17. The method according to claim 16, wherein triolein is sourced from high oleic sunflower oil.

18. The method according to any one of claims 10 to 13, wherein the method comprises mixing a second fatty acid with the second triglyceride, the first fatty acid, and the modified enzyme, and the first triglyceride is a mixture of fatty acids from the second triglyceride, the first fatty acid and the second fatty acid, wherein the first fatty acid is different from the second fatty acid.

19. The method according to claim 18, wherein the first triglyceride is 1-oleoyl-2-palmitoyl-3-linoleoylglycerol (OPL), the second triglyceride is tripalmitin, the first fatty acid is oleic acid, and the second fatty acid is linoleic acid.

20. The method according to any one of claim 18 or 19, wherein a weight ratio of the first fatty acid to the second fatty acid is from 1:9 to 9:1.

21. The method according to claim 15, or claim 19, or claim 20, wherein tripalmitin is sourced from a fractionated fraction of palm oil enriched in tripalmitin.

22. A method of producing biodiesel, the method comprises mixing a fatty acid or a triglyceride, a short-chained alcohol, and the modified enzyme according to any one of claims 1 to 9 under suitable conditions to form a fatty acid ester of the short-chained alcohol.

23. The method according to claim 22, wherein the short-chained alcohol is selected from the group consisting of methanol, ethanol, n-propanol and isopropanol.

24. The method according to claim 22 or 23, wherein the fatty acid or the triglyceride is one or more selected from crude palm oil, sludge oil, fatty matter, used or unused cooking oil, palm fatty acid distillate, tallow, and brown grease.
